(19) United States
(12) Patent Application Publication
(10) Pub. No.: US 2024/0182603 A1
(43) Pub. Date: Jun. 6, 2024

(54) SULFUR-SUBSTITUTED SUGAR TO STABILIZE OLIGOSACCHARIDE

(71) Applicant: Brandeis University, Waltham, MA (US)

(72) Inventors: Isaac J. Krauss, Waltham, MA (US); Leiming Tian, Waltham, MA (US)

(21) Appl. No.: 18/551,000

(22) PCT Filed: Mar. 18, 2022
(86) PCT No.: PCT/US22/20849

§ 371 Date: Sep. 18, 2023

Related U.S. Application Data
(60) Provisional application No. 63/163,668, filed on Mar. 19, 2021.

(51) Int. Cl. C08B 37/00; C07H 21/00; G01N 33/68

(52) U.S. Cl. CPC C08B 37/006; C07H 21/00; G01N 33/6854; G01N 2400/02; G01N 2400/38

(57) ABSTRACT

Glycosylated peptides and oligonucleotides of the invention contain oligosaccharides that include three or more saccharide moieties, wherein two saccharide moieties at a non-reducing terminal end of the oligosaccharide are coupled together with a thio-ether bond, and one of the saccharide moieties at a reducing end of the oligosaccharide is coupled to a reactive moiety. Also disclosed are immunogenic conjugates that include a glycopeptide or oligonucleotide bound to an immunogenic carrier molecule, as well as pharmaceutical compositions containing the same.

This application claims the priority benefit of U.S. Provisional Patent Application Ser. No. 63/163,668, filed Mar. 19, 2021, which is hereby incorporated by reference in its entirety.

This invention was made with government support under Grant Nos. R01 AI090745 and R01 AI113737 awarded by the National Institutes of Health. The government has certain rights in this invention.

### FIELD OF THE INVENTION

This invention relates to oligosaccharides that include two saccharide moieties at a non-reducing terminal end of the oligosaccharide that are coupled together with a thio-ether bond, as well as oligosaccharide-decorated peptides and oligonucleotides, immunogenic conjugates and pharmaceutical compositions containing the same, and their use to induce immune responses against the same.

### BACKGROUND OF THE INVENTION

Carbohydrate or glycoconjugate vaccines (Lang et al., “Carbohydrate Conjugates in Vaccine Developments,” Front. Chem. 8:284 (2020)) are in use or development for prevention of bacterial infections (Finn, “Bacterial Polysaccharide-Protein Conjugate Vaccines,” Br. Med. Bull. 70:1-14 (2004); Rappuoli, “Glycoconjugate Vaccines: Principles and mechanisms,” Sci. Transl. Med. 10 (2018)), cancer (Heimburg-Molinaro et al., “Cancer Vaccines and Carbohydrate Epitopes,” Vaccine 29:8802-26 (2011)), and HIV (Horiya et al., “Recent Strategies Targeting HIV Glycans in Vaccine Design,” Nat. Chem. Biol. 10:990-9 (2014); Liu et al., “Broadly Neutralizing Antibody-Guided Carbohydrate-Based HIV Vaccine Design: Challenges and Opportunities,” Chem Med Chem 11:357-62 (2016); Bastida et al., “Synthetic Carbohydrate-based HIV-1 Vaccines,” Drug Discov Today Technol 35-36:45-56 (2020)). In HIV vaccine development, there is significant interest in elicitation of antibodies that can bind to the Manα1→2Man moieties of high mannose (Man₉GlcNAc₂) glycans (Seabright et al., “Protein and Glycan Mimicry in HIV Vaccine Design,” J. Mol. Biol. 431:2223-2247 (2019); Calarese et al., “Antibody Domain Exchange Is an Immunological Solution to Carbohydrate Cluster Recognition,” Science 300:2065 (2003); Pejchal et al., “A Potent and Broad Neutralizing Antibody Recognizes and Penetrates the HIV Glycan Shield,” Science 334:1097-1103 (2011); Kong et al., “Supersite of Immune Vulnerability on the Glycosylated Face of HIV-1 Envelope Glycoprotein gp120,” Nat. Struct. Mol. Biol. 20:796-803 (2013)).

However, it has been shown that, for glycoconjugate vaccines, mannosidase trimming degrades this motif so that the antibody response is directed against the glycan core or other structures in the glycoconjugate (Nguyen et al., “Oligomannose Glycopeptide Conjugates Elicit Antibodies Targeting the Glycan Core Rather than Its Extremities,” ACS Cent Sci 5:237-249 (2019); Bruxelle et al., “Serum Alpha-mannosidase as an Additional Barrier to Eliciting Oligomannose-specific HIV-1-neutralizing Antibodies,” Sci. Rep. 10:7582 (2020)). It would be desirable, therefore, to develop glycoconjugate vaccines that are stabilized against enzymatic hydrolysis.

The present invention is directed to overcoming these and other deficiencies in the art.

### SUMMARY OF THE INVENTION

A first aspect of the invention relates to an oligosaccharide that includes three or more saccharide moieties, wherein two saccharide moieties at a non-reducing terminal end of the oligosaccharide are coupled together with a thio-ether bond, and one of the saccharide moieties at a reducing end of the oligosaccharide is coupled to a reactive moiety.

A second aspect of the invention relates to a glycopeptide that includes an oligosaccharide covalently linked to a sidechain of at least one amino acid residue in the peptide, the oligosaccharide comprising two or more saccharide moieties coupled together with a thio-ether bond.

A third aspect of the invention relates to a glycosylated oligonucleotide molecule that includes at least one non-natural deoxynucleoside to which is covalently linked a branched or unbranched oligosaccharide comprising two or more saccharide moieties coupled together with a thio-ether bond.

The glycopeptide and glycosylated oligonucleotide according to the second and third aspect of the invention can be prepared by reacting the reactive moiety of the oligosaccharide to the sidechain of the at least one amino acid residue in the peptide or the non-natural deoxynucleoside in the oligonucleotide molecule.

A fourth aspect of the invention relates to an immunogenic conjugate that includes a glycopeptide according to the second aspect or the oligonucleotide according to the third aspect, which is bound to an immunogenic carrier molecule, wherein the glycopeptide or oligonucleotide binds specifically to a carbohydrate-binding monoclonal antibody.

A fifth aspect of the invention relates to a pharmaceutical composition that includes an immunogenic conjugate according to fourth aspect, glycopeptide according to the second aspect or the oligonucleotide according to the third aspect.

A sixth aspect of the invention relates to a method of detecting a carbohydrate-binding antibody that includes the steps of: contacting a sample with a glycopeptide according to the second aspect or the oligonucleotide according to the third aspect; and detecting whether a carbohydrate-binding antibody in the sample binds to the glycopeptide or the oligonucleotide.

A seventh aspect of the invention relates to a method of inducing an immune response in an individual that includes the step of: administering to an individual a glycopeptide according to the second aspect, the oligonucleotide according to the third aspect, or the immunogenic conjugate according to the fourth aspect, wherein said administering is effective to induce an immune response against the glycopeptide or the oligonucleotide.

An eighth aspect of the invention relates to a method of treating a cancerous condition that includes the step of: administering to an individual a glycopeptide according to the second aspect, the oligonucleotide according to the third aspect, or the immunogenic conjugate according to the fourth aspect, wherein said administering is effective to induce an anti-tumor immune response against a cancer cell expressing a glycosylated cancer-specific protein.

A ninth aspect of the invention relates to an intermediate disaccharide having the structure:

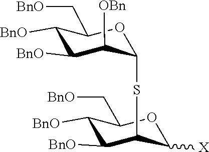

where X is a leaving group, and is either in an alpha or beta orientation.

A tenth aspect of the invention relates to a method of preparing a reactive oligosaccharide that includes the step of reacting the intermediate disaccharide according to the ninth aspect of the invention with an acceptor monosaccharide or oligosaccharide that is partially protected and comprises at least one unprotected hydroxyl group, said reacting being carried out under conditions suitable to cause the disaccharide to covalently bond to the at least one unprotected hydroxyl groups to form an oligosaccharide according to the first aspect of the invention.

By selecting for glycopeptides and glycosylated oligonucleotides that are capable of binding to neutralizing antibodies, or antibody binding fragments thereof, that are known to bind to carbohydrate-decorated antigen, the glycopeptides and glycosylated oligonucleotides of the present invention can be used to mimic the native carbohydrate-decorated antigen associated with infectious agents, such as viruses, and cancer cells. Thus, pharmaceutical compositions containing the glycopeptides, glycosylated oligonucleotides, or immunogenic conjugates of the present invention can be used to inhibit infection or proliferation of virus particles or inhibit cancer cell proliferation.

In the accompanying Examples, a disaccharide donor containing a thio-ether linkage (see 1, FIG. 1) was prepared and its ability to participate in stereospecific glycosylation with anchimeric assistance from the thioether linkage was assessed. Glycosyl donors containing simple 2-thio substituents are known (Hashimoto et al., “A Stereocontrolled Construction of 2-deoxy-β-glycosidic Linkages via 1,2-trans-β-glycosidation of 2-deoxy-2-[(p-methoxyphenyl)thio]glycopyranosyl N,N,N′,N′-tetramethylphosphoroamidates,” Chem. Lett. 1511-14 (1992); Toshima et al., “Highly β-stereoselective Glycosylation by Use of 1-O-acetyl-2,6-anhydro-2-thio Glycosyl Donor for Synthesis of 2,6-dideoxy-β-glycosides,” Tetrahedron Lett. 33: 1491-4 (1992); Toshima et al., “Novel Glycosidation Method Using 2,6-Anhydro-2-thio Sugars for Stereocontrolled Synthesis of 2,6-Dideoxy-α- and -β-glycosides,” J. Am. Chem. Soc. 116:9042-51 (1994); Toshima et al., “Application of Highly Stereocontrolled Glycosidations Employing 2,6-Anhydro-2-thio Sugars to the Syntheses of Erythromycin A and Olivomycin A Trisaccharide,” J. Am. Chem. Soc. 117:3717-27 (1995); Roush et al., “Stereoselective Preparation of 2-deoxy-β-glycosides from Glycal Precursors—2. Stereochemistry of Glycosidation Reactions of 2-thiophenyl- and 2-selenophenyl-α-D-glucopyranosyl Donors,” Tetrahedron 53:8837-8852 (1997); Castro-Palomino et al., “Synthesis of Ganglioside GD3 and Its Comparison with Bovine GD3 with Regard to Oligodendrocyte Apoptosis Mitochondrial Damage,” Chem.—Eur. J. 7:2178-2184 (2001); Knapp et al., “Glycosylation with 2′-thio-S-acetyl Participation,” Tetrahedron Lett. 44:7601-7605 (2003); Shirahata et al., “Improved Catalytic and Stereoselective Glycosylation with Glycosyl N-trichloroacetylcarbamate: Application to Various 1-hydroxy Sugars,” Carbohydr. Res. 345:740-749 (2010), each of which is hereby incorporated by reference in its entirety), but only one thio-linked disaccharide donor has been reported, with a gluco-configuration (Hashimoto et al., “Synthesis of α-L-fucopyranosyl Disaccharides with Thioglycosidic Linkages and Characterization of α-L-fucosidases from Bovine Kidney and Epididymis By Their Inhibitory Activities,” Tetrahedron: Asymmetry 5:2351-66 (1994), which is hereby incorporated by reference in its entirety). Glycosylation with dimannose derivative 1 would offer an efficient route to serum-stabilized fragments of Man₉GlcNAc₂, or potentially the whole oligosaccharide. While antibodies raised against some S-linked glycan analogs exhibit cross-reactivity with the natural oxygen-linked sugars (Bundle et al., “Thiooligosaccharide Conjugate Vaccines Evoke Antibodies Specific for Native Antigens,” Angew Chem Int Ed Engl 44:7725-9 (2005); Rich et al., “S-linked Ganglioside Analogues for use in Conjugate Vaccines,” Org. Lett. 6:897-900 (2004); Wu et al., “Synthesis and Immunochemical Characterization of S-linked Glycoconjugate Vaccines Against Candida albicans,” Chem. Eur. J. 14: 6474-82 (2008); Huo et al., “Synthetic and Immunological Studies of N-acyl Modified S-linked STn Derivatives as Anticancer Vaccine Candidates,” Org. Biomol. Chem. 13:3677-90 (2015); Kuan et al., “Synthesis of an S-Linked alpha(2→8) GD3 Antigen and Evaluation of the Immunogenicity of Its Glycoconjugate,” Chem. Eur. J. 23:6876-6887 (2017), each of which is hereby incorporated by reference in its entirety), such analogs have not previously been tested in the case of oligomannose vaccines.

### BRIEF DESCRIPTION OF THE DRAWINGS

FIG. 1 illustrates a synthetic scheme for thioether-linkage-assisted stereospecific glycosylation.

FIG. 2 illustrates a synthetic scheme for synthesis of an S-linked disaccharide donor.

FIG. 3 illustrates a synthetic scheme for synthesis of S-linked Man₃.

FIG. 4 illustrates a synthetic scheme for synthesis of S-linked Man₄.

FIGS. 5A-B illustrate the strain-promoted azide/alkyne cycloaddition with DBCO amine linker 23 in order to facilitate separation and detection of degradation products by LC/MS. The scheme for preparation of 23 is shown in FIG. 5A. The LC/MS results are illustrated collectively in FIG. 5B.

FIG. 6A-B illustrates the binding analysis of S-Man₄ to HIV broadly neutralizing antibody 2G12. FIG. 6A shows the STD-NMR spectrum of S-Man₄ (21) with 2G12 IgG (upper spectrum). The bottom spectrum (blue) shows the reference 800 MHz ¹H NMR whereas the top (red) shows corresponding STD spectrum. Numbers indicate selected assignments by carbon number and ring letter. FIG. 6B shows the crystal structure for all O-linked Man₄ (22) bound to 2G12 (PDB ID 6MSY).

FIG. 7 shows the STD-NMR spectrum of for S-Man₃ (14) with HIV IgG antibody 2G12. The bottom spectrum (blue) shows the reference 800 MHz ¹H NMR whereas the top (red) shows corresponding STD spectrum. Numbers indicate selected assignments by carbon number and ring letter.

FIG. 8A-B show SPAAC labeling (FIG. 8A) and LC/MS results showing extent of mannosidase digestion of O-linked Man₄ (22) (FIG. 8B).

FIGS. 9A-B show SPAAC labeling (FIG. 9A) and LC/MS results showing extent of mannosidase digestion of S-Man₄ (21) (FIG. 9B).

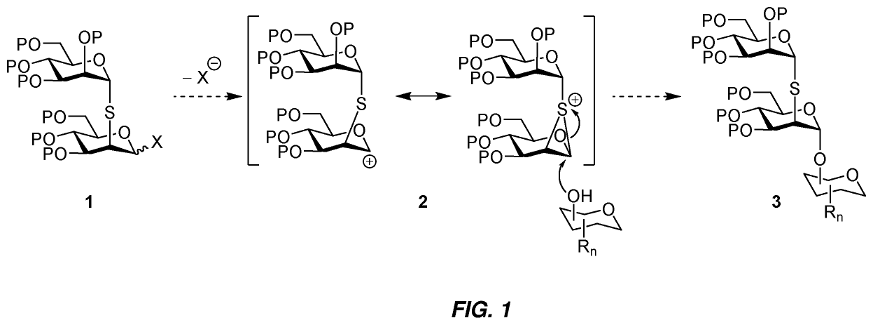

### DETAILED DESCRIPTION OF THE INVENTION

Disclosed herein are oligosaccharides that include two saccharide moieties at a non-reducing terminal end of the oligosaccharide that are coupled together with a thio-ether bond. These oligosaccharides can be used to form oligosaccharide-decorated peptides and oligonucleotides, immunogenic conjugates and pharmaceutical compositions containing the same, and these can be use to induce immune responses against the oligosaccharide-decorated peptides and oligonucleotides which mimic oligosaccharide-containing antigen.

The oligosaccharides of the invention include three or more saccharide moieties, wherein two saccharide moieties at a non-reducing terminal end of the oligosaccharide are coupled together with a thio-ether bond, and one of the saccharide moieties at a reducing end of the oligosaccharide is coupled to a reactive moiety.

The reactive moiety allows the oligosaccharides to be coupled covalently to the oligonucleotide or peptide backbone, preferably by “click” chemistry reaction between the reactive moiety and a modified nucleoside or modified amino acid molecule. Exemplary reactive moieties include, without limitation, an azido group, an alkynyl group, an alkenyl group, a thiol group, an amino group, or a carboxylic acid group. Appropriate click chemistry reactions include thiol-ene reactions (reaction of a thiol bond across an alkene or alkyne by either a free radical or ionic mechanism) (see, e.g., Hoyle et al., Angew. Chem. Int. Ed. 49:1540-1573 (2010), which is hereby incorporated by reference in its entirety) as well as azide-alkyne cycloaddition reactions (reaction of an azido group with a terminal or internal alkyne) (see, e.g., Temme et al., Chem. Eur. J. 19:17291-17295 (2013) and Hong et al., Angew. Chem. Int. Ed. 48:9879-9883 (2009), which are hereby incorporated by reference in their entirety). Typically, copper catalysis or ruthenium catalysis or strain-promoted alkyne-azide cycloaddition is used.

The oligosaccharides may include a linker between the one saccharide moiety and the reactive moiety. Exemplary linker groups include, without limitation, —C₃-C₈ cycloalkyl-, —(CH₂)ₙ—, —S—(CH₂)ₙ—, —O—(CH₂)ₙ—, —(CH₂)ₘ—S—(CH₂)ₙ—, —(CH₂)ₘ—O—(CH₂)ₙ—, —O-aryl, or —S-aryl, wherein n and m are independently an integer from 1 to 20, preferably 2 to 10 or 2 to 4.

The oligosaccharides may contain from three to twenty saccharide moieties, such as from three to ten saccharide moieties, from eleven to twenty saccharide moieties, from four to twelve saccharide moieties, or from four to ten saccharide moieties. Also encompassed are oligosaccharides that contain three saccharide moieties, four saccharide moieties, five saccharide moieties, six saccharide moieties, seven saccharide moieties, eight saccharide moieties, nine saccharide moieties, ten saccharide moieties, eleven saccharide moieties, twelve saccharide moieties, thirteen saccharide moieties, fourteen saccharide moieties, fifteen saccharide moieties, sixteen saccharide moieties, seventeen saccharide moieties, eighteen saccharide moieties, nineteen saccharide moieties, or twenty saccharide moieties.

The oligosaccharides may be branched or unbranched. For branched oligosaccharides, the branched oligosaccharide may contain a thio-ether bond between two saccharide moieties at a non-reducing terminal end of each branch in the oligosaccharide. Thus, a branched oligosaccharide containing two branches may contain a thio-ether bond between two saccharide moieties at a non-reducing terminal end of both branches. Similarly, a branched oligosaccharide containing three branches may contain a thio-ether bond between three saccharide moieties at a non-reducing terminal end of all three branches.

In the oligosaccharides, the saccharide moieties can be glucose, galactose, mannose, arabinose, fucose, rhamnose, sialic acid, N-acetyl-glucosamine, as well as any combinations thereof.

Non-limiting examples of oligosaccharides that are capable of “click” reaction with alkenyl or alkynyl groups (present in amino acid sidechains or modified nucleoside bases) include, without limitation:

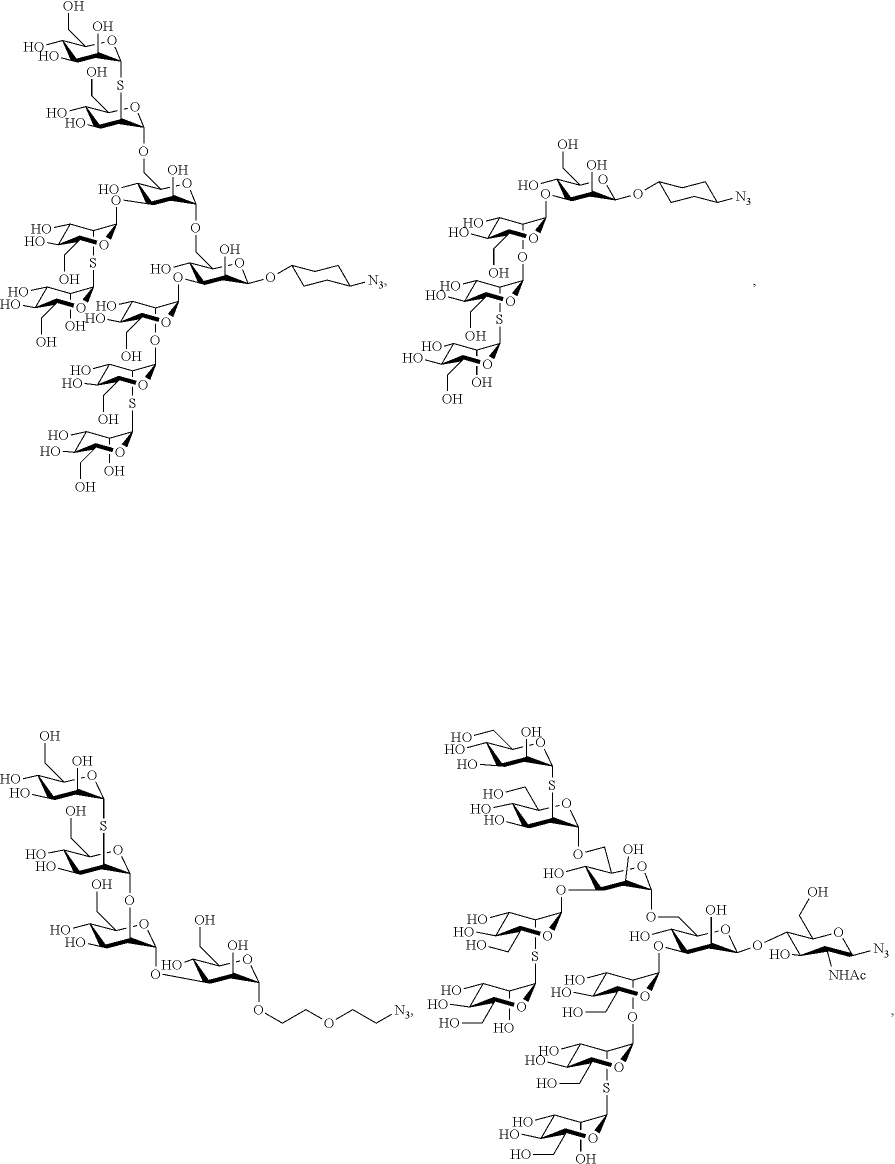

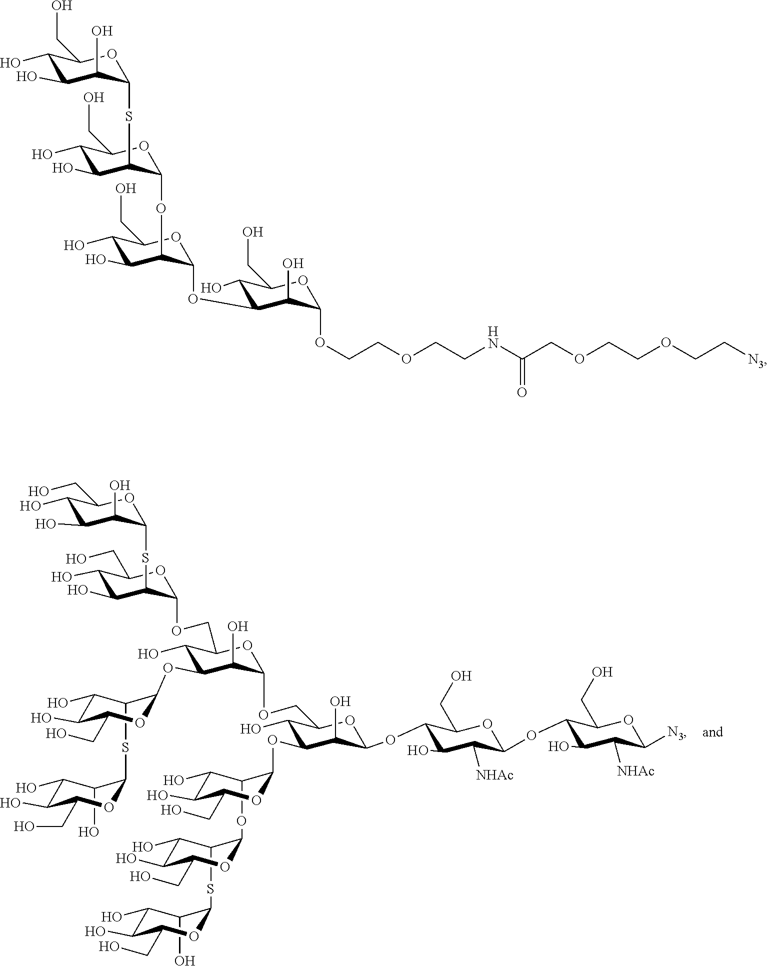

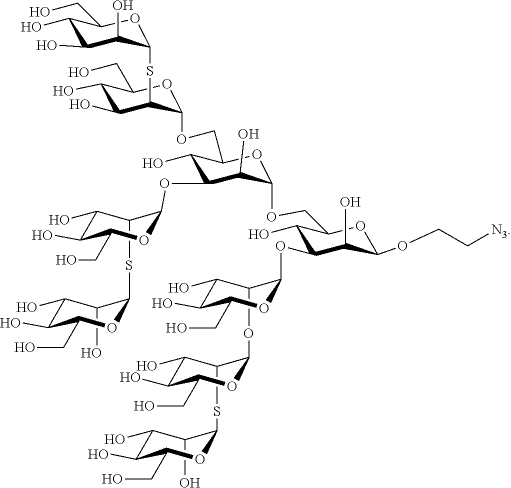

Corresponding structures with the azido group replaced by an alkenyl or alkynyl group are also contemplated.

The thio-linked oligosaccharides of the present invention can be prepared using the procedures described in the accompanying Examples. Briefly, a protected, thio-ether donor dissacharide is reacted under suitable conditions with an acceptor monosaccharide or oligosaccharide, which may or may not be branched, such that the donor disaccharide forms the two saccharide moieties present at the one or more non-reducing terminal end(s) of the oligosaccharide. The acceptor monosaccharide or oligosaccharide is partially protected, which directs the acceptor to react with unprotected hydroxyl groups. Exemplary conditions include, without limitation, anhydrous dichloromethane solvent, and cooling to about −50° C. with addition of Hf(OTf)₄ and stirring for a sufficient period of time, and then the reaction can be neutralized with trimethylamine. The recovered, protected oligosaccharide can be purified and later deprotected and optionally further purified as desired.

The donor disaccharide according to one embodiment has the structure:

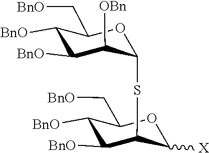

where X is a leaving group, and is either in an alpha or beta orientation. Exemplary leaving groups include, without limitation, F, Cl, Br, I, and sulfonates such as p-toluenesulfonate (OTs) and methanesulfonate (OMs). Other leaving groups can also be utilized. This donor disaccharide can be prepared according to the procedures described in Example 1.

Having obtained a thio-linked oligosaccharides of the present invention, the oligosaccharide is ready to be linked, for example, using appropriate “click” chemistry, to peptide sidechains or to nucleoside bases, which affords the glycosylated peptides or oligonucleotides of the present invention.

According to one embodiment, the glycopeptides of the present invention can be prepared using the in vitro method for selection described in PCT Application Publ. No. WO/2015/084867, which is hereby incorporated by reference in its entirety. Briefly, using a carbohydrate binding antibody, preferably a neutralizing monoclonal antibody, allows for the selection of glycopeptides that mimic the native antigen that the antibody binds. The selection process involves glycopeptides presented in an mRNA display with unnatural amino acids incorporated into the glycopeptides for “click” chemistry coupling of the thio-ether protected oligosaccharides as described herein.

The method for selecting a glycopolypeptide that binds to a target protein includes providing a pool of glycopolypeptides fused via puromycin linker to an encoding mRNA-cDNA duplex; combining the pool with a target protein to form a mixture; incubating the mixture for a period of time sufficient to allow any target protein to bind to one or more of the glycopolypeptides, thereby forming glycopolypeptide-target protein complexes; and isolating from the mixture the glycopolypeptide-target protein complexes, thereby identifying a plurality of selected glycopolypeptides. Multiple rounds of selection and regenerating mRNA-linked glycopolypeptide pools can be performed in the manner illustrated in PCT Application Publ. No. WO/2015/084867, which is hereby incorporated by reference in its entirety.

The provided pool of glycopolypeptides fused via puromycin linker to an encoding mRNA-cDNA duplex is preferably large enough to afford sufficient diversity so as to allow for selection of multiple, diverse glycopolypeptides that exhibit target protein binding capability. By way of example, the provided pool comprises about 10¹⁰ or greater, about 10¹¹ or greater, about 10¹² or greater, or about 10¹³ or greater glycopolypeptides fused via puromycin linker to an encoding mRNA-cDNA duplex.

Creation of the first pool is carried out by first generating a library of DNA duplexes of sufficient length to afford a glycopeptide pool of the desired complexity. Each DNA duplex includes a promoter sequence to allow for transcription, optionally an enhancer element sequence, a sequence containing a ribosomal binding site that affords in vitro translation of mRNA transcripts, an open reading frame region that affords sequence variety to generate glycopolypeptide diversity, and a downstream sequence that encodes, e.g., a His tag followed by a constant region that serves as the linker for puromycin. Any suitable promoter and enhancer sequences suitable for in vitro transcription can be used, and any suitable ribosomal binding sequence can be used. Sequence variation can be introduced using random diversity at each site or semi-random diversity at each site.

The generation of pools of mRNA-supported glycopolypeptides and selection of individual pool members against target proteins, preferably a carbohydrate-binding monoclonal antibody, is demonstrated in PCT Application Publ. No. WO/2015/084867, which is hereby incorporated by reference in its entirety. The DNA duplexes are used as templates for generating mRNA templates. This can be achieved using any suitable in vitro transcription protocol. Thereafter, a puromycin linker is attached to the 3′ region of the mRNA strand. Briefly, purified transcripts can be photo-crosslinked with puromycin-containing oligonucleotide. Photo-crosslinking is achieved using, e.g., 365 nm UV irradiation as previously described (Kurz et al., Nucleic Acids Res. 28:e83 (2000) and Seelig, B. Nat. Protocols 6:540-552 (2011), which are hereby incorporated by reference in their entirety).

Use of puromycin at the 3′ region of the mRNA transcript allows for mRNA-display of the translated polypeptide based on the physical linkage of the polypeptide to the mRNA that encoded it. Puromycin inhibits translation by mimicking the substrate of the ribosome—the 3′ end of an aminoacyl-tRNA. As ribosomes complete the translation of individual mRNAs to the corresponding peptides they encounter the 3′ puromycin. Because puromycin is chemically similar to the 3′ end of aminoacyl-tRNA, it is recognized by the peptidyl transfer center of the ribosome, which catalyzes the transfer of the nascent polypeptide to the modified tyrosine of puromycin. The mRNA is now covalently attached to the corresponding translated peptide via the puromycin, and the ribosomes are stalled. To promote the covalent attached (or fusion) of the translated polypeptide to the encoding mRNA strand, the reaction mixture is preferably exposed to KCl and Mg(OAc)₂ and then maintained at a temperature below 0° C. for sufficient duration to yield the fused product. At this point, the initial pool or library mRNAs have now been translated and linked via puromycin to the peptides that they encode in a stable molecular conjugate referred to as an mRNA-peptide fusion.

To facilitate glycosylation of the translated polypeptide, translation of the mRNA strand is carried out using one or more modified amino acids comprising a reactive side chain. One exemplary amino acid is homopropargylglycine, which is efficiently recognized for incorporation into the polypeptide corresponding to the location of Met codons. Thus, for purposes of translation, homopropargylglycine constitutes a modified methionine. Homopropargylglycine can be prepared using the procedures of Shimizu et al., Nat Biotech 19:751-755 (2001); Josephson et al., J. Am. Chem. Soc. 127:11727-11735 (2005); Guillen et al., J. Am. Chem. Soc. 134:10469-10477 (2012); Shimizu et al., Methods Mol Biol. Vol. 607, p 11-21 (2010); and Ma et al., Ribosome Display and Related Technologies; Douthwaite, J. A., Jackson, R. H., Eds.; Springer New York: Methods Mol Biol. Vol. 805, p 367-390 (2012), which are hereby incorporated by reference in their entirety. Other exemplary amino acids are p-azido-phenylalanine and p-ethynyl-phenylalanine, which are efficiently recognized for incorporation into the polypeptide corresponding to the location of Phe codons when the PheRS A294G substrate is used (see Hartmann et al., PlosOne DOI 10.1371/journal.pone.0000972 (2007), which is hereby incorporated by reference in its entirety. Yet another exemplary amino acid is L-allyl glycine which is efficiently recognized for incorporation into the polypeptide corresponding to the location of Leu codons when the editing deficient LeuRS D345A substrate is used. With modified amino acylated-tRNAs introduced into the reaction mixture in the absence of one or more natural amino acylated-tRNAs, the modified amino acids are introduced into the polypeptide chain (Guillen et al., J. Am. Chem. Soc. 134:10469-10477 (2012), which is hereby incorporated by reference in its entirety).

The resulting translated polypeptide can include any number of amino acids, preferably between about 10 to about 80 amino acids, more preferably between about 15 and about 70 amino acids. In certain embodiments, the polypeptide can include about 20 amino acids, about 25 amino acids, about 30 amino acids, about 35 amino acids, about 40 amino acids, about 45 amino acids, about 50 amino acids, about 55 amino acids, about 60 amino acids, or about 65 amino acids. The polypeptide can include one or more of the modified amino acid residues, preferably between about 2 to about 10 of the modified amino acid residues. In certain embodiments, the polypeptide can include 2 to 5 modified amino acids, or 6 to 10 modified amino acids.

The modified amino acids can be located at adjacent positions (i.e., where one modified amino acid is linked via peptide bond to another modified amino acid) or at nonadjacent positions (i.e., where no two modified amino acids are linked via peptide bond to one another). In certain embodiments, the resulting polypeptide includes a plurality of modified amino acids, some of which are adjacent to one another and some of which are not adjacent to another modified amino acid.

After forming the mRNA-polypeptide fusion, the one or more monosaccharides or oligosaccharides are attached using appropriate click chemistry reactions, which include thiol-ene reactions (reaction of a thiol bond across an alkene or alkyne by either a free radical or ionic mechanism) as well as azide-alkyne cycloaddition reactions (reaction of an azido group with a terminal or internal alkyne) as described above. As a result of the click reaction between the modified amino acid and the oligosaccharide, the glycopolypeptide contains a linker molecule between the polypeptide chain and the oligosaccharide. Exemplary linker molecules include, without limitation:

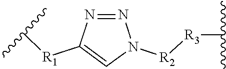

(resulting from the azide-alkyne reaction) or

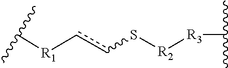

(resulting from the alkene/alkyne-thiol reaction), wherein each of R₁ and R₂ is optionally a direct link or independently selected from the group consisting of a linear or branched C₁ to C₁₈ hydrocarbon that is saturated or mono- or poly-unsaturated, optionally interrupted by one or more non-adjacent —O—, —C(═O)—, or —NR₄—; a substituted or unsubstituted C₃ to C₁₀ cycloalkandiyl, a substituted or unsubstituted aryl diradical; a substituted or unsubstituted heteroaryl diradical; a monosaccharide diradical; or a disaccharide diradical; R₃ is optional and can be —O—, —S—, or —NR₄—; and R₄ is H or a C₁ to C₁₀ alkyl. Although flexible linkers may be used, the linker between the oligosaccharide and the modified amino acid(s) of the glycopeptide preferably includes one or more cyclic moieties which offer some rigidity to the resulting glycosyl group.

The polypeptide may optionally include two or more cysteine residues that allow for cyclization via bis-alkylbenzene or bis-alkylpyridine linkers. This is described in PCT Patent Application Publ. No. WO 2020/086885, which is hereby incorporated by reference in its entirety.

According to another embodiment, a glycopeptide of the present invention can be prepared by synthesizing a specific peptide sequence using solid-phase synthesis. Residues that are to be coupled with the oligosaccharide, such as homopropargylglycine, L-allyl glycine, p-azido-phenylalanine, or p-ethynyl-phenylalanine as described above, can be introduced during solid-phase synthesis at desired locations. After recovery of the synthesized peptide, glycosylation of the peptide at one or more locations can be carried out using appropriate “click” chemistry reactions for covalent attachment of the oligosaccharides.

The glycopolypeptides of the present invention include one or more of the modified amino acid residues having a sidechain comprising an oligosaccharide as described above, and the glycopolypeptide binds specifically to a carbohydrate-binding monoclonal antibody with an affinity of less than 100 nM.

In certain embodiments, the glycopolypeptide binds specifically to the carbohydrate-binding monoclonal antibody with an affinity (K_{d}) of less than 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, or 1 nM.

In preferred embodiments, the glycopolypeptide binds specifically to the carbohydrate-binding monoclonal antibody with an affinity that is substantially the same as or lower than the affinity of the carbohydrate-binding monoclonal antibody to its naturally occurring binding partner. As used herein, an affinity that is “substantially the same” means that as Kd of glycopeptide for its target is less than 5×, less than 4×, less than 3×, less than 2×, or less than 1.5×Kd of the native binding partner to the monoclonal antibody. In certain embodiments, the glycopolypeptide binds specifically to the carbohydrate-binding monoclonal antibody with an affinity that is lower than the affinity of the carbohydrate-binding monoclonal antibody to its naturally occurring binding partner.

Exemplary carbohydrate-binding neutralizing monoclonal antibodies include those that bind specifically to N-glycosylated HIV gp120 or N-glycosylated HSV-2 gD. Specific examples of these neutralizing monoclonal antibodies include, without limitation, 2G12, PG9, PG16, PGT121, PGT122, PGT123, PGT125, PGT126, PGT127, PGT128, PGT129, PGT130, PGT131, PGT135, PGT136, PGT137, PGT141, PGT142, PGT143, PGT144, PGT145, PGT151, PGT152, PGT153, PGT154, PGT155, PGT156, PGT157, PGT158, CH01, CH02, CH03, CH04, 10-1074, 10-996, 10-1146, 10-847, 10-1341, 10-1121, 10-1130, 10-410, 10-303, 10-259, 10-1369, and E317.

Exemplary carbohydrate-binding cytotoxic monoclonal antibodies include those that binds specifically to O-glycosylated cancer-specific human podoplanin; aberrantly O-glycosylated cancer-specific MUC1, aberrantly O-glycosylated cancer-specific Integrin α3β1, or N-glycosylated cancer-specific antigen RAAG12. Specific examples of these cytotoxic monoclonal antibodies include, without limitation, LpMab-2 (Kato et al., Sci Rep. 4:5924 (2014), which is hereby incorporated by reference in its entirety), 237 MAb (Brooks et al., PNAS 107(22):10056-10061 (2010), which is hereby incorporated by reference in its entirety), RAV12 (Loo et al., Mol. Cancer Ther. 6(3):856-65 (2007), which is hereby incorporated by reference in its entirety), BCMab1 (Clinical Cancer Research 20(15):4001 (2014), which is hereby incorporated by reference in its entirety), DF3 and 115D8 (Tang et al., Clin Vaccine Immunol. 17(12): 1903-1908 (2010), which is hereby incorporated by reference in its entirety), huHMFG1, HT186-B7, -D11 and -G2 sc-FVs (Thie et al., PLoS One 6(1): e15921 (2011), which is hereby incorporated by reference in its entirety), and GOD3-2C4 (Welinder et al. Glycobiol. 21(8):1097-107 (2011), which is hereby incorporated by reference in its entirety).

According to one embodiment, the glycosylated oligonucleotides of the present invention can be prepared using the in vitro method for selection described in U.S. Patent Application Publ. No. 20130116417 or PCT Application Publ. No. WO 2015/084846, which is hereby incorporated by reference in its entirety. Briefly, using a carbohydrate binding antibody, preferably a neutralizing monoclonal antibody, allows for the selection of glycosylated oligonucleotides that mimic the native antigen that the antibody binds. The selection process involves selection of glycosylated oligonucleotides containing one or more modified nucleoside bases incorporated into the oligonucleotides for “click” chemistry coupling of the thio-ether protected oligosaccharides as described herein.

Accordingly, the method for selecting a glycosylated oligonucleotide that binds to a target protein includes providing a pool of modified, single-strand-double-strand hybrid oligonucleotides that are glycosylated within the single-strand region, combining the pool with a target protein to form a mixture, incubating the mixture at a temperature above 20° C. for a period of time sufficient to allow any target protein to bind one or more of the modified, single-strand-double-strand hybrid oligonucleotides, and isolating from the mixture the modified, single-strand-double-strand hybrid oligonucleotides that bind to the target protein, thereby identifying a plurality of selected oligonucleotides. Multiple rounds of selection and regenerating pools of modified, single-strand-double-strand hybrid oligonucleotides that are glycosylated within the single-strand region can be performed in the manner illustrated in PCT Application Publ. No. WO/2015/084846, which is hereby incorporated by reference in its entirety.

The selection procedure uses diverse DNA backbones to cluster the glycans in various ways (MacPherson et al., Angew. Chem. Int. Ed. 50:11238-11242 (2011); Temme et al., Chem. Eur. J. 19:17291-17295 (2013), which are hereby incorporated by reference in their entirety). The library is constructed using copper assisted alkyne/azide cycloaddition (CuAAAC) chemistry (Kolb et al., Angew. Chem. Int. Ed. 40:2004-2021 (2001); Rostovtsev et al., Angew. Chem. Int. Ed. 41:2596-2599 (2002); Gierlich et al., Org. Lett. 8:3639-3642 (2006); Gierlich et al., Chem. Eur. J. 13:9486-9494 (2007), which are hereby incorporated in their entirety) to attach glycans to a library of random DNA sequences containing alkynyl bases. In a single-stranded portion of the modified, single-strand-double-strand hybrid oligonucleotides, each DNA sequence clusters the glycans in a unique geometry, and the clusters which are selected from the library by binding to the target protein (e.g., neutralizing monoclonal antibody) are amplified by PCR to generate a new library for further selection. The process is then repeated for several cycles with increasingly stringent selection conditions.

The provided pool of modified, single-strand-double-strand hybrid oligonucleotides that are glycosylated within the single-strand region is preferably large enough to afford sufficient diversity so as to allow for selection of multiple, diverse oligonucleotides that exhibit target protein binding capability. By way of example, the provided pool comprises about 10¹⁰ or greater, about 10¹¹ or greater, about 10¹² or greater, or about 10¹³ or greater modified, single-strand-double-strand hybrid oligonucleotides that are glycosylated within the single-strand region.

Creation of the first pool is carried out by first generating a library of single-stranded, hairpin-loop DNA structures of sufficient length to afford an oligonucleotide of the desired complexity. Each hairpin-loop structure includes a loop portion having a primer binding site (discussed infra) and a partial stem portion that includes a region at the 3′ end hybridized to a complementary region displaced from the 5′ end. Initially, the sequence between the complementary region and the 5′ terminus includes a random sequence. This random sequence can be of any length suitable to afford introduction of one or more glycans, typically from about 15 to about 100 nucleobases in length, more preferably about 15 to about 60 nucleobases or about 20 to about 50 nucleobases in length.

In certain embodiments, the region containing the randomized sequence comprises from about 10% to about 20% adenine (A). Preferably, the randomized region comprises about 10%, about 11%, about 12%, about 13%, about 14%, about 15%, about 16%, about 17%, about 18%, about 19%, or about 20% adenine (A). More preferably, the randomized region comprises 15% adenine (A).

To facilitate glycosylation of the resulting oligonucleotide, the initial library is treated with a polymerase, dNTPs and modified dNTPs or modified rNTPs, under conditions effective to allow for extension of the 3′ end using the sequence between the complementary region and the 5′ terminus as a template. The resulting stem-loop nucleic acid includes a single strand containing one or more modified nucleosides (near the 3′ end). Any nucleoside base that contains a reactive group suitable for click chemistry coupling of a compatibly modified monosaccharide or oligosaccharide to the oligonucleotide can be used during this extension step. Examples of modified nucleosides that can be introduced during this extension step include, without limitation, N₆-(6-azido)hexyl-dATP (Jena Bioscience), C8-alkyne-dCTP (Jena Bioscience), 5-ethynyl-dUTP (Jena Bioscience), C8-alkyne-dUTP (Jena Bioscience), 5-azido-C₃-UTP (Jena Bioscience), 5-ethynyl-UTP (Jena Bioscience), N⁶ -propargyl-ATP (Jena Bioscience), 2-ethynyl-ATP (Jena Bioscience), and 8-azido-ATP. As a consequence of introducing these modified nucleosides to form the 3′ end extension, this portion of the strand, containing the one or more modified nucleosides, has one or more azido or alkynyl groups (alkenyl groups can also be used) available for click reaction.

The modified nucleosides can be located at adjacent positions (i.e., where one modified nucleosides is linked via the sugar-phosphate backbone to another modified nucleoside) or at nonadjacent positions (i.e., where no two modified nucleosides are linked via the sugar-phosphate backbone to one another). In certain embodiments, the resulting oligonucleotide includes a plurality of modified nucleosides, some of which are adjacent to one another and some of which are not adjacent to another modified nucleoside.

After introducing the modified nucleosides to the 3′ extension, the one or more monosaccharides or oligosaccharides are attached using appropriate click chemistry reactions, which include thiol-ene reactions as well as azide-alkyne cycloaddition reactions as described above. As a result of the click reaction between the modified nucleoside base and the oligosaccharide, the glycosylated oligonucleotide contains a linker molecule between the oligonucleotide chain and the oligosaccharide. Exemplary linker molecules include those described above for the glycopolypeptides.

According to another embodiment, a glycosylated oligonucleotide of the present invention can be prepared by solid-phase synthesis. Modified nucleoside bases that are to be coupled with the oligosaccharide, such as N₆-(6-azido)hexyl-dATP, C8-alkyne-dCTP, 5-ethynyl-dUTP, C8-alkyne-dUTP, 5-azido-C₃-UTP, 5-ethynyl-UTP, N⁶-propargyl-ATP, 2-ethynyl-ATP, and 8-azido-ATP as described above, can be introduced during solid-phase synthesis at desired locations. After recovery of the synthesized oligonucleotide, glycosylation of the oligonucleotide at one or more locations can be carried out using appropriate “click” chemistry reactions for covalent attachment of the oligosaccharides.

In one embodiment, the oligonucleotide comprises from 2 to about 10 of said modified nucleoside bases. In certain embodiments, the oligonucleotide may contain from 2 to 5 or 2 to 4 of the modified nucleoside bases, and preferably two or more of the modified nucleoside bases are at nonadjacent positions in the oligonucleotide. In certain embodiments, two modified nucleoside bases may be at adjacent positions in the oligonucleotide.

In certain embodiments, the oligonucleotide binds specifically to the carbohydrate-binding monoclonal antibody with an affinity (K_{d}) of less than 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, or 1 nM.

In certain embodiments, the oligonucleotide binds specifically to the carbohydrate-binding monoclonal antibody with an affinity that is substantially the same as or lower than the affinity of the carbohydrate-binding monoclonal antibody to its naturally occurring binding partner. As used herein, an affinity that is “substantially the same” means that a Kd of the oligonucleotide for its target monoclonal antibody is less than 5×, less than 4×, less than 3×, less than 2×, or less than 1.5×Kd of the native binding partner to the monoclonal antibody. In certain embodiments, the oligonucleotide binds specifically to the carbohydrate-binding monoclonal antibody with an affinity that is lower than the affinity of the carbohydrate-binding monoclonal antibody to its naturally occurring binding partner.

A further aspect of the invention relates to an immunogenic conjugate that includes a glycopolypeptide or glycol-oligonucleotide of the invention covalently or non-covalently bound to an immunogenic carrier molecule. Exemplary immunogenic carrier molecule include, without limitation, bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein.

Any of a variety of conjugation methodologies can be utilized. See, e.g., Jennings et al., J. Immunol. 127:1011-8 (1981); Beuvery et al., Infect. Immun 40:39-45 (1993), each of which is hereby incorporated by reference in its entirety. In one approach terminal aldehyde groups can be generated through periodate oxidation, and the aldehydes are then reacted through reductive amination with free amino groups on the protein, mostly lysines, in the presence of sodium cyanoborohydride. In another approach, a carbodiimide reaction is performed to covalently link carboxylic groups to the lysine ε-amino groups on the carrier protein. The activation sites in this method are more random, compared to periodate activation.

A further aspect of the invention relates to a pharmaceutical composition that includes a pharmaceutically acceptable carrier and a glycopolypeptide, glycol-oligonucleotide, or immunogenic conjugate of the invention.

Pharmaceutical compositions suitable for injectable or parental use (e.g., intravenous, intra-arterial, intramuscular, etc.) or intranasal use may include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. Suitable adjuvants, carriers and/or excipients, include, but are not limited to sterile liquids, such as water, saline solutions, and oils, with or without the addition of a surfactant and other pharmaceutically and physiologically acceptable carriers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

The pharmaceutical compositions of the present invention may also be administered directly to the airways in the form of an aerosol. For use as aerosols, the compositions of the present invention in the form of a solution or suspension may be packaged in a pressurized aerosol container together with suitable propellants, for example, hydrocarbon propellants like propane, butane, or isobutane with conventional adjuvants. The pharmaceutical compositions of the present invention also may be administered in a non-pressurized form such as in a nebulizer or atomizer. Formulations suitable for intranasal nebulization or bronchial aerosolization delivery are also known and can be used in the present invention (see Lu & Hickey, “Pulmonary Vaccine Delivery,” Exp Rev Vaccines 6(2):213-226 (2007) and Alpar et al., “Biodegradable Mucoadhesive Particulates for Nasal and Pulmonary Antigen and DNA Delivery,” Adv Drug Deliv Rev 57(3):411-30 (2005), which are hereby incorporated by reference in their entirety.

The pharmaceutical compositions of the present invention can also include an effective amount of a separate adjuvant. Suitable adjuvants for use in the present invention include, without limitation, aluminum hydroxide, aluminum phosphate, aluminum potassium sulfate, beryllium sulfate, silica, kaolin, carbon, water-in-oil emulsions, oil-in-water emulsions, muramyl dipeptide, bacterial endotoxin, lipid, Quil A, non-infective Bordetella pertussis, QS-21, monophosphoryl lipid A, an alpha-galactosylceramide derivative, or PamCys-type lipids.

The choice of an adjuvant depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, alum, MPL or Incomplete Freund's adjuvant (Chang et al., Advanced Drug Delivery Reviews 32:173-186 (1998), which is hereby incorporated by reference in its entirety) alone or optionally all combinations thereof are suitable for human administration.

The pharmaceutical compositions can also include one or more additives or preservatives, or both.

Effective amounts of the glycopolypeptide or glyco-oligonucleotide may vary depending upon many different factors, including mode of administration, target site, physiological state of the patient, other medications administered, and whether treatment is prophylactic or therapeutic. Treatment dosages need to be titrated to optimize safety and efficacy. The amount of glycopolypeptide or glyco-oligonucleotide immunogen depends on whether adjuvant is also administered, with higher dosages being required in the absence of adjuvant. The amount of a glycopolypeptide or glyco-oligonucleotide immunogen for administration sometimes varies from 1 μg-5 mg per patient and more usually from 5-1000 μg per injection for human administration.

The glycopolypeptides, glyco-oligonucleotides, immunogenic conjugates, and pharmaceutical compositions can be incorporated into a delivery vehicle to facilitate administration. Such delivery vehicles include, but are not limited to, biodegradable microspheres (MARK E. KEEGAN & W. MARK SALTZMAN, Surface Modified Biodegradable Microspheres for DNA Vaccine Delivery, in DNA VACCINES: METHODS AND PROTOCOLS 107-113 (W. Mark Saltzman et al., eds., 2006), which is hereby incorporated by reference in its entirety), microparticles (Singh et al., “Nanoparticles and Microparticles as Vaccine Delivery Systems,” Expert Rev Vaccine 6(5):797-808 (2007), which is hereby incorporated by reference in its entirety), nanoparticles (Wendorf et al., “A Practical Approach to the Use of Nanoparticles for Vaccine Delivery,” J Pharmaceutical Sciences 95(12):2738-50 (2006) which is hereby incorporated by reference in its entirety), liposomes (U.S. Patent Application Publication No. 2007/0082043 to Dov et al. and Hayashi et al., “A Novel Vaccine Delivery System Using Immunopotentiating Fusogenic Liposomes,” Biochem Biophys Res Comm 261(3): 824-28 (1999), which are hereby incorporated by reference in their entirety), collagen minipellets (Lofthouse et al., “The Application of Biodegradable Collagen Minipellets as Vaccine Delivery Vehicles in Mice and Sheep,” Vaccine 19(30):4318-27 (2001), which is hereby incorporated by reference in its entirety), and cochleates (Gould-Fogerite et al., “Targeting Immune Response Induction with Cochleate and Liposome-Based Vaccines,” Adv Drug Deliv Rev 32(3):273-87 (1998), which is hereby incorporated by reference in its entirety).

The glycopolypeptides, glyco-oligonucleotides, immunogenic conjugates, and pharmaceutical compositions can be used to induce an immune response in an individual. The individual can be any mammal, particularly a human, although veterinary usage is also contemplated. This method is carried out by administering one of these active agents to an individual in a manner that is effective to induce an immune response against the glycopolypeptide or glyco-oligonucleotide. Because the glycopolypeptide or glyco-oligonucleotide mimics the native glycosylated epitope of a native target of the monoclonal antibody to which the glycopolypeptide or or glyco-oligonucleotide was selected, certain glycopolypeptides and/or glyco-oligonucleotides can induce a carbohydrate-binding, neutralizing antibody response that is protective against a pathogen (e.g., viral or bacterial pathogen) and certain other glycopolypeptides and/or glyco-oligonucleotides can induce a carbohydrate-binding, cytotoxic antibody response against a cancer cell that expresses a glycosylated antigen.

For each of these embodiments, administration of the glycopolypeptides, glyco-oligonucleotides, immunogenic conjugates, and/or pharmaceutical compositions can be carried orally, parenterally, subcutaneously, intravenously, intramuscularly, intraperitoneally, by intranasal instillation, by implantation, by intracavitary or intravesical instillation, intraarterially, intralesionally, transdermally, intra- or peri-tumorally, by application to mucous membranes, or by inhalation. Administration of these agents can be repeated periodically.

Exemplary viruses include, without limitation, Calicivirus, Chikungunya virus, Cytomegalovirus, Dengue virus, Eastern Equine Encephalitis virus, Ebola virus, Epstein-Barr virus, Hantaan virus, Hepatitis A virus, Hepatitis B virus, Hepatitis C virus, Hepatitis D virus, Hepatitis E virus, Herpes simplex virus, Human Immunodeficiency virus (HIV-1 or HIV-2), Human Papillomavirus, Influenza virus, Japanese encephalitis virus, Junin virus, Lassa virus, Marburg virus, Measles virus, Metapneumovirus, Nipah virus, Newcastle disease virus, Norwalk virus, Parainfluenza virus, Poliovirus, Rabies virus, Respiratory Syncytial virus, Rift Valley Fever virus, Rotavirus, Rubella virus, Sendai virus, Severe Acute Respiratory Syndrome (SARS Co-V), Tick-borne Encephalitis virus, Varicella zoster virus, Venezuelan Equine Encephalitis virus, Yellow Fever virus, Western Equine Encephalitis virus, and West Nile virus.

Exemplary bacteria include, without limitation, Bacillus anthracis, Bordetella pertussis B, Borrelia burgdorferi, Chlamydia trachomatis, Clostridium difficile, Clostridium tetani, Candida albicans, Corynebacterium diphtherias, Cryptococcus neoformans, Entamoeba histolytica, Escherichia coli, Francisella tularensis, Haemophilus influenzae (nontypeable), Helicobacter pylori, Histoplasma capsulatum, Moraxella catarrhalis, Mycobacterium leprae, Mycobacterium tuberculosis, Neisseria gonorrheae, Neisseria meningitides, Pseudomonas aeruginosa, Staphylococcus aureus, Methicillin-resistant Staphylococcus aureus, Streptococcus agalactiae, Streptococcus pneumoniae, Streptococcus pyogenes, and Yersinia pestis.

For prophylactic treatment against viral or bacterial infection, it is intended that the glycopolypeptides, glyco-oligonucleotides, immunogenic conjugates, and pharmaceutical compositions of the present invention can be administered prior to exposure of an individual to the virus or bacteria and that the resulting immune response can inhibit or reduce the severity of the viral or bacterial infection such that the virus or bacteria can be eliminated from the individual. The glycopolypeptides, glyco-oligonucleotides, immunogenic conjugates, and pharmaceutical compositions of the present invention can also be administered to an individual for therapeutic treatment. In accordance with one embodiment, it is intended that the composition(s) of the present invention can be administered to an individual who is already exposed to the virus or bacteria. The resulting enhanced immune response can reduce the duration or severity of the existing viral or bacterial infection, as well as minimize any harmful consequences of untreated viral or bacterial infections. The composition(s) can also be administered in combination other therapeutic anti-viral or anti-bacterial regimen. In asymptomatic patients, treatment can begin at any age (e.g., 10, 20, 30 years of age). Treatment typically entails multiple dosages over a period of time. Treatment can be monitored by assaying antibody, or activated T-cell or B-cell responses to the therapeutic agent over time. If the response falls, a booster dosage is indicated.

The glycopolypeptides, glyco-oligonucleotides, immunogenic conjugates, and pharmaceutical compositions that induce a cytotoxic antibody response against a cancer cell antigen can be used to treat solid tumors and blood cancers (leukemia or lymphoma) that are characterized by expression of O-glycosylated cancer-specific human podoplanin; aberrantly O-glycosylated cancer-specific MUC1, aberrantly O-glycosylated cancer-specific integrin α3β1, or N-glycosylated cancer-specific antigen RAAG12.

Exemplary cancers that display one of the glycosylated cancer-specific antigen include colorectal cancer, gastric cancer, ovarian cancer, breast cancer, and pancreatic cancer, which display N-glycosylated RAAG12; squamous cell carcinoma, lung and esophageal carcinoma, testicular seminoma, malignant brain tumor, fibrosarcoma, malignant mesothelioma, bladder cancers, and testicular cancers that display O-glycosylated ppodoplanin; bladder cancers that display O-glycosylated integrin α3β1; breast cancer, ovarian cancer, lung cancer, pancreatic cancer, prostate cancer, and forms of leukemia that displays aberrantly O-glycosylated MUC1.

For cancer therapy, it is contemplated that the glycopolypeptides, glyco-oligonucleotides, immunogenic conjugates, and pharmaceutical compositions can be administered in combination with a chemotherapeutic agent, a radiation therapy, or alternative immunotherapeutic agent. The specific selection of chemotherapeutic agent, a radiation therapy, or alternative immunotherapeutic agent will depend on the type of cancer. These agents can also be administered in combination with surgical resection to remove cancerous tissue, with treatment being carried out before, after, or both before and after surgery.

For inducing the immune response, the amount of a glycopolypeptide or glyco-oligonucleotide for administration sometimes varies from 1 μg-5 mg per patient and more usually from 5-1500 μg per dose for human administration. Occasionally, a higher dose of 1-2 mg per injection is used. Typically about 10, 20, 50, or 100 μg is used for each human dose. The mass of the glycopolypeptide or glyco-oligonucleotide immunogen also depends on the mass ratio of immunogenic epitope within the glycopolypeptide or glyco-oligonucleotide immunogen to the mass of glycopolypeptide or glyco-oligonucleotide immunogen as a whole. Typically, 10⁻³ to 10⁻⁵ micromoles of immunogenic epitope are used for each microgram of glycopolypeptide or glyco-oligonucleotide immunogen. The timing of injections can vary significantly from once a day, to once a year, to once a decade. On any given day that a dosage of glycopolypeptide or glyco-oligonucleotide immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster administration at time intervals, such as 6 week intervals. Another regimen consists of an immunization followed by booster injections 1, 2, and 12 months later. Another regimen entails an administration every two months for a prolonged period in excess of 12 months. Alternatively, booster injections can be on an irregular basis as indicated by monitoring of immune response.

In certain embodiments, multiple doses are given over a period of time, each using a different immunogenic glycopolypeptide or glycol-oligonucleotide in an appropriate amount, as indicated above.

The glycopolypeptides or glyco-oligonucleotides of the invention can also be used to detect a neutralizing antibody in a patient sample (e.g., a serum sample). This method includes providing a glycopolypeptide or glyco-oligonucleotide of the invention, contacting the glycopolypeptide or glyco-oligonucleotide with a sample from an individual; and detecting whether the glycopolypeptide or glyco-oligonucleotide binds specifically to an antibody present in the sample, wherein the detection of the antibody is carried out using a label.

Exemplary labels include, without limitation, a radiolabel, fluorescent label, enzymatic label, chemiluminescent marker, biotinyl group, an epitope recognized by a secondary reporter, a magnetic agent, or a toxin.

The detection step is preferably carried using a suitable assay format. Exemplary assays include, without limitation, ELISA, radioimmunoassay, gel-diffusion precipitation reaction assay, immunodiffusion assay, agglutination assay, fluorescent immunoassay, immunoelectrophoresis assay, surface plasmon resonance assay, or biolayer interferometry assay. In certainly of these assay formats, a secondary antibody is used to label the antibody bound specifically to the glycopolypeptide. Depending on the type of assay, the glycopolypeptide can be in the solution phase or coupled to a solid surface.

### EXAMPLES

The examples below are intended to exemplify the practice of the present invention but are by no means intended to limit the scope thereof.

### Materials and Methods

Reactions were carried out in oven-dried glassware under a nitrogen atmosphere and were stirred magnetically. All reagents were purchased from Sigma-Aldrich, Acros Organics, Fluka, Alfa Aesar or TCI, and used without further purification unless otherwise noted. Molecular sieves were crushed and freshly activated prior to use by heating on gas flame in quartz round bottom flask under <1 Torr on a vacuum manifold attached to a Welch 1400 vacuum pump. The word “concentrated” refers to removal of solvents by means of rotary evaporator attached to a Welch 1400 oil pump (bled to 5-300 mm Hg as needed) followed by removal of residual solvents at <1 Torr on a vacuum manifold attached to a Welch 1400 vacuum pump. SiliaFlash® F60 (230-400 mesh) from Silicycle® was used for flash column chromatography unless specifically indicated.

Analytical thin layer chromatography (TLC) was performed using silica gel F-354 pre-coated glass plates (0.25 mm). TLC plates were analyzed by short wave UV illumination or dipping in cerium-ammonium-molybdate (CAM) stain (40 g of ammonium pentamolybdate, 1.6 g of Cerium (IV) sulfate, 800 mL of diluted sulfuric acid (1:9, with water, v/v) and heating on a hot plate.

Tetrahydrofuran (THF), dichloromethane (DCM) and toluene, were purified by degassing with argon and passage through activated alumina columns.

¹H, ¹³C, ¹⁹F and HSQC NMR were recorded on a Varian Innova 400 MHz and Bruker 800 MHz in deuterated solvents at ambient temperature unless otherwise noted. Spectra acquired in CDCl₃ were internally referenced to TMS (0 ppm) or residual CDCl₃ (7.26 for ¹H and 77.06 for ¹³C). ¹⁹F Fluorine spectra were referenced to trifluorotoluene (−63.72 ppm), internally or by substituting a tube containing standard. Spectra acquired in D₂O were referenced to sodium 3-(trimethylsilyl) propane sulfonate by the substitution method, or referenced to the HDO peak using temperature-specific values. Chemical shifts are reported in parts per million (ppm). Coupling constants are reported in Hz with multiplicities denoted as s (singlet), d (doublet), dd (doublet of doublets), t (triplet), q (quartet), m (multiplate), app (apparent) and br (broad). High Resolution mass spectra (HRMS) were performed by Mass Spectrometry Laboratory, University of Illinois at Urbana-Champaign using electron impact (EI) or electrospray (ESI) ionization and a TOF analyzer. LC/MS analysis was performed at Brandeis University on a Waters Acquity UPLC chromatograph with reverse phase C18 and C4 column, and Waters Photodiode Array and Micro mass ZQ4000 mass detectors. IR spectra were recorded on a Varian 640-IR FT-IR spectrometer and are reported in wavenumbers (cm⁻¹). Optical rotation was measured on JASCO-polarimeter.

### Example 1—Synthesis of Thio-Disaccharide Donor

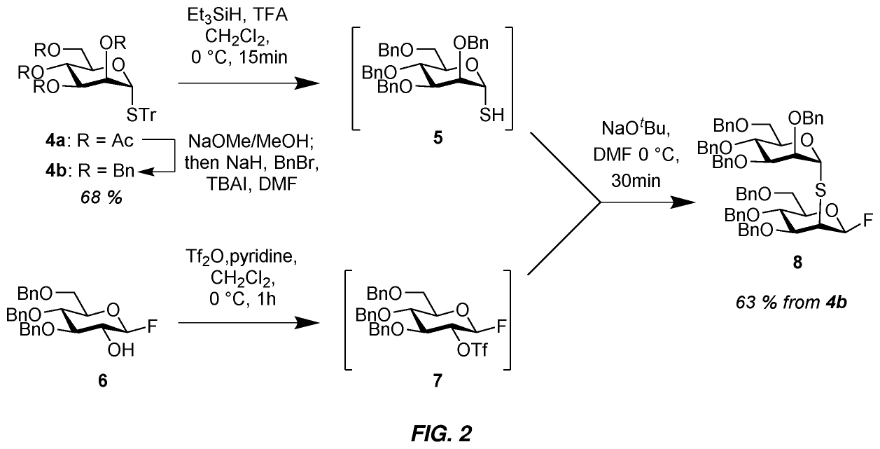

To prepare the requisite thio-disaccharide donor, the known trityl thioglycoside 4a was used as a starting material (FIG. 2) (Matta et al., “Synthesis of p-nitrobenzyl and p-nitrophenyl 1-thioglycopyranosides,” Carbohydr. Res. 43:101-9 (1975); Belz et al., “A Building Block Approach to the Synthesis of a Family of S-linked alpha-1,6-oligomannosides,” Carbohydr. Res. 429:38-47 (2016), each of which is hereby incorporated by reference in its entirety). Exchange to benzyl protecting groups proceeded in 68% overall yield to afford building block 4b. It was then evaluated whether 5-derived thiolate could displace a 2-triflate derivative with a relatively inert leaving group such as a fluoride already present at C1.

Thus, 1-fluoro glucose derivative 6 was prepared by a known protocol including epoxidation of tribenzyl glucal (Cheshev et al., “Direct Epoxidation of D-glucal and D-galactal Derivatives with in situ Generated DMDO,” Carbohydr. Res. 341:2714-6 (2006), which is hereby incorporated by reference in its entirety), followed by TBAF treatment (Gordon et al., “Displacement Reactions of a 1,2-anhydro-alpha-D-hexopyranose: Installation of Useful Functionality at the Anomeric Carbon,” Carbohydr. Res. 206:361-6 (1990), which is hereby incorporated by reference in its entirety). Following triflation of 6 and triethylsilane/trifluoroacetic acid deprotection of 4b, 5 and 7 were combined and allowed to react in the presence of sodium tert-butoxide to afford the desired disaccharide 8 in 63% yield.

Individual reaction steps and evaluation by ¹H, ¹³C, ¹⁹F and/or HSQC NMR are described below.

### Triphenylmethyl 2,3,4,6-tetra-O-benzyl-1-thio-α-D-mannopyranoside (4b)

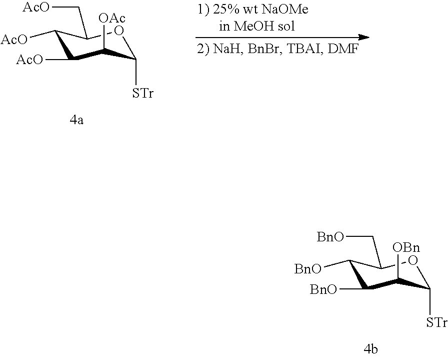

NaOMe in methanol (25% wt; 565 μL; 2.47 mmol; 0.3 equiv.) was added to a solution of mannoside 4a (Matta et al., “Synthesis of p-nitrobenzyl and p-nitrophenyl 1-thioglycopyranosides,” Carbohydr Res 43:101-9 (1975), which is hereby incorporated by reference in its entirety) (5 g; 8.24 mmol; 1 equiv.) dissolved in dry dichloromethane and methanol (1:1; 50 mL) at rt with stirring for 4 h. The solution was adjusted to pH 6-7 with Amberlite IR-120 resin (H⁺ form), filtered, and the filtrate evaporated to dryness under reduced pressure to give the tetraol, which was coevaporated with toluene two times and used without purification for next step. Crude tetraol (3.6 g; 8.21 mmol; 1 equiv.) was dissolved in dry DMF (30 ml) and cooled in ice bath. Sodium hydride (1.97 g; 49.25 mmol; 6 equiv; 60%) was added to reaction mixture and stirred for 30 minutes at 0° C., followed by dropwise addition of benzyl bromide (5.85 mL; 49.25 mmol; 6 equiv.) and TBAI (2.1 g; 2.21 mmol; 1 equiv.). The reaction mixture was stirred for 6 hours at rt and quenched with 2 mL of methanol at 0° C. The reaction mixture was then diluted with EtOAc and washed with brine, then dried over MgSO₄, filtered and the solvent evaporated under reduced pressure. The crude was purified by column chromatography (1:5; EtOAc:hexane) to give compound 4b as a yellow syrup (4.4 g; 5.58 mmol) in 68% yield.

¹H NMR (400 MHz, CDCl₃) δ 7.50-7.10 (multiple signals, 35H+residual CHCl₃), 4.87 (d, J=10.8 Hz, 1H), 4.83 (d, J=1.6 Hz, 1H), 4.69 (d, J=12.1 Hz, 1H), 4.58-4.41 (m, 4H), 4.25-4.17 (m, 1H), 4.15 (d, J=12.6 Hz, 1H), 3.99 (t, J=9.6 Hz, 1H), 3.89-3.70 (m, 4H), 3.61 (app. t, J=2.4 Hz, 1H). ¹³C NMR (100 MHz, CDCl₃, Selected signals): δ 144.7, 138.6, 138.4, 138.3, 138.2, 130.1, 128.3, 128.2, 128.1, 127.9, 127.9, 127.8, 127.6, 127.5, 127.4, 126.9, 81.8, 80.4, 78.1, 75.1, 74.6, 74.5, 71.9, 70.6, 69.8, 69.3. [α]_{D}²⁵: +76.1 (c1.0, CHCl₃). IR (cm⁻¹): 3028, 2863, 1949, 1451, 1087. HRMS (ESI-TOF) m/z: [M+Na]⁺ Calcd. for C₅₃H₅₀O₅SNa: 821.3277, found 821.3294.

### 2,3,4,6-Tetra-O-benzyl-α-D-mannopyranosyl thiol (5)

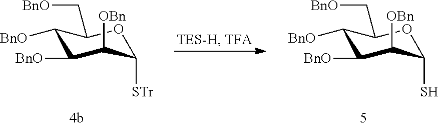

Triethyl silane (1.5 mL; 9.39 mmol; 5 equiv.) and TFA (3 mL, 30.04 mmol; 16 equiv.) were added to a solution of mannosyl trityl thiol 4 (1.5 g; 1.88 mmol; 1 equiv.) dissolved in dry dichloromethane (30 mL) under a N₂ atm. After 30 minutes the reaction had reached completion and the solvents were evaporated followed by co-evaporation with toluene and kept for high vacuum for 3 hours to afford crude of compound 5 as yellow oil, which was used in next steps without purification.

¹H NMR (400 MHz, CDCl₃) δ 7.67-6.76 (multiple signals, 20H+residual CHCl₃+ triphenylmethane), 5.66 (dd, J=7.0, 1.8 Hz, 1H), 4.87 (d, J=10.7 Hz, 1H), 4.71 (d, J=12.5 Hz, 1H), 4.67-4.45 (multiple signals, 6H), 4.11 (ddd, J=9.4, 5.0, 1.8 Hz, 1H), 4.01 (app t, J=9.4 Hz, 1H), 3.86 (dd, J=9.4, 3.0 Hz, 1H), 3.81 (dd, J=3.0, 1.8 Hz, 1H), 1H), 3.68 (dd, J=10.8, 1.8 Hz, 1H), 2.02 (d, J=7.0 Hz, 1H). ¹³C NMR (101 MHz, CDCl₃, Selected Signals) δ 138.2, 138.1, 138.0, 137.8, 128.3, 128.3, 128.3, 128.2, 128.2, 127.9, 127.8, 127.8, 127.8, 127.7, 127.7, 127.6, 127.5, 127.5, 79.0, 75.1, 76.2, 74.8, 73.3, 72.5, 72.1, 72.0, 68.9, 67.9, 56.8. HRMS (ESI-TOF) m/z: [M+Na]⁺: Calcd. for C₃₄H₃₆O₅SNa: 579.2181, found 579.2173.

### 3,4,6-Tri-O-benzyl-β-D-glucopyranosyl fluoride (6)

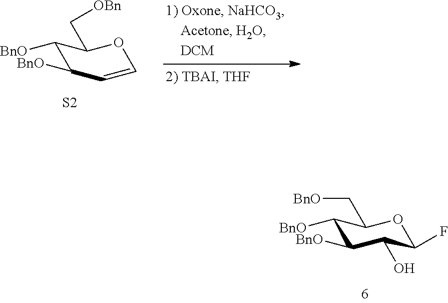

To a vigorously stirred, cooled biphasic solution of 3,4,6-tri-O-benzyl-D-glucal S2 (Cheshev et al., “Direct Epoxidation of D-glucal and D-galactal Derivatives with in situ Generated DMDO,” Carbohydr Res 341:2714-6 (2006), which is hereby incorporated by reference in its entirety) (6 g, 14.42 mmol; 1 equiv.) in dichloromethane (60 mL), acetone (6 mL) and sat. aq. NaHCO₃ (100 mL), a solution of oxone (17.74 g; 57.68 mmol; 4 equiv.) dissolved in water (36 mL) was added dropwise over 20 minutes. The reaction mixture was stirred vigorously for 2 hours at room temperature. The organic layer was separated, and aqueous layer was extracted twice with 140 mL of dichloromethane. The combined organic layers were dried by MgSO₄ and concentrated under reduced pressure to afford white solid 1,2-anhydro-3,4,6-tri-O-benzyl-α/β-D-glucopyranose, which was used for next reaction without purification and dissolved in anhydrous THF (60 mL) and cooled to 0° C. 1M Tetrabutylammonium fluoride in THF³ (28.2 mL; 28.21 mmol; 2 equiv.) was added dropwise and stirred for 6 hours. The reaction mixture was extracted with dichloromethane 150 mL two times and combined organic layers were dried over MgSO₄, filtered and concentrated under reduced pressure. The crude was purified by column chromatography (1:4; EtOAc:hexane) to afford compound 6 (1.8 g; 8.32 mmol) as yellow amorphous solid in 29% yield over two steps.

¹H NMR (400 MHz, CDCl₃) δ 7.42-7.12 (multiple signals, 15H+residual CHCl₃), 5.14 (dd, ¹J_{FH}=53.1, J_{H1H2}=6.4 Hz, 1H), 4.81 (app s, 2H), 4.76 (d, J=10.9 Hz, 1H), 4.59 (d, J=12.1 Hz, 1H), 4.56 (d, J=˜11 Hz, 1H), 4.53 (d, J=12.1 Hz, 1H), 3.80-3.63 (m, 5H), 3.58 (app t, J=8.2 Hz, 1H), 2.56 (d, J=3.9 Hz, 1H). ¹³C NMR (101 MHz, CDCl₃) δ 138.1, 137.8, 137.6, 128.5, 128.4, 128.4, (127.92, 127.91, 127.84, 127.82, 127.71, 6 resonances with an overlap), 109.0 (C—F, d, ¹J_{CF}=215.1 Hz), 81.1 (C—F d, ³J_{CF}=9.3 Hz), 76.3, 74.86, 74.82, 74.5, 73.5, 73.0 (C—F, d, ²J_{CF}=23.7 Hz), 68.48. ¹⁹F NMR (376 MHz, CDCl₃) δ −140.13 (dd, J_{HF}=53.2, 11.7). [α]_{D}²⁵: +59.76 (c1.0, CHCl₃). IR (cm⁻¹):3431.3, 2872.1, 2362.2, 2019.1, 1736.4, 1080.1, 1027.7. HRMS (ESI-TOF) m/z: [M+Na]⁺: Calcd. for C₂₇H₂₉FO₅Na: 475.1897, found 475.1898.

### 3,4,6-tri-O-benzyl-2-O-trifluoromethylsulfonyl-β-D-glucopyranosyl fluoride (7)

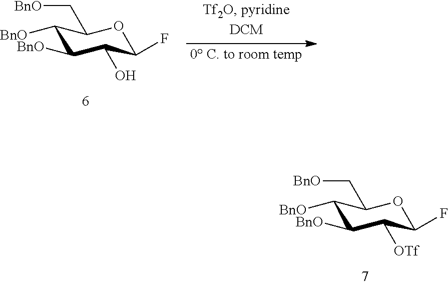

To a solution of compound 6 (1 g; 2.21 mmol; 1 equiv.) and pyridine (2.19 mL, 22.1 mmol, 10 equiv.) in 15 mL of CH₂Cl₂ was added Tf₂O (408 μL; 2.43 mmol; 1.1 equiv.) at 0° C. under N₂ atm. After 0.5 hour at 0° C. and 1 h at ambient temperature, the solution was diluted with 30 mL of CH₂Cl₂ and washed with ice-cold sat aq. NaHCO₃, ice-cold solution of CuSO₄, and brine. The organic layer was then dried over MgSO₄, filtered and concentrated in vacuo to afford 1.21 g (2.06 mmol; 93%) of compound 7 as yellow liquid which was used for next reaction without further purification.

¹H NMR (400 MHz, CDCl₃) δ 7.34-7.12 (multiple signals, 15H+residual CHCl₃), 5.301 (dd, ¹J_{FH}=52.4 Hz, J_{H1H2}=7.0 Hz, 1H), 4.87-4.70 (multiple signals, 4H), 4.60-4.48 (multiple signals, 3H), 3.84 (app t, J=9.1 Hz, 1H), 3.77 (app t, J=9.1 Hz, 1H), 3.739-3.668 (m, 2H), 3.61 (ddd, J=9.4 Hz, 2.9 Hz, 2.9 Hz, 1H). ¹³C NMR (100 MHz, CDCl₃) δ 137.27, 136.98, 136.56, (128.31, 128.26, 127.89, 127.67, 127.65, 127.62; 9 resonances with overlaps), 118.17 (C—F, q, ¹J_{CF}=319.54 Hz), 105.15 (C—F, d, ¹J_{CF}=219.07 Hz), 83.90 (C—F, d, ²J_{CF}=24.7 Hz), 80.32 (C—F, d, ³J_{CF}=8.4 Hz), 75.56, 75.14 (C—F, d, long range J_{CF}=4.3 Hz), 74.89, 73.40, 67.38. ¹⁹F NMR (376 MHz, CDCl₃) δ −74.38 (d, ⁶J_{FF}=9.7 Hz), −140.11 (ddq, ¹J_{HF}=52.4, ²J_{HF}=9.7 Hz, ⁶J_{FF}=9.7 Hz). LRMS (ESI-single quad) m/z: [M+NH₄]⁺: Calcd. for C₂₈H₃₂F₄O₇NS: 602.1836, Found: 602.15 (this compound was too unstable to send for HRMS).

### 3,4,6-tri-O-benzyl-2-S-(2,3,4,6-tetra-O-benzyl-α-D-mannopyranosyl-(1→2))-β-D-mannopyranosyl fluoride (8)

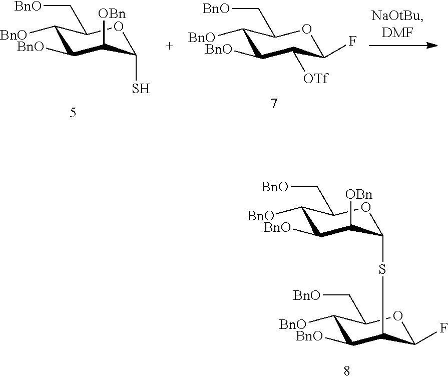

To a solution of freshly prepared crude compound 5 and compound 7 (1.21 g; 2.07 mmol; 1.1 equiv.) in 20 mL of dry DMF was added NaO^{t}Bu (181 mg; 1.89 mmol; 1 equiv.) under N₂ atm. After 30 min, the reaction mixture was diluted with 50 mL of EtOAc and washed with ice-cold brine solution. Combined organic layers were dried over MgSO₄, filtered and evaporated under reduced pressure maintaining water bath temperature 30° C. The crude was purified by column chromatography by using triethyl amine quenched silica gel (1:5; EtOAc:hexane) to afford compound 8 (1.18 g; 1.19 mmol) as yellow amorphous solid in 63% yield.

¹H NMR (400 MHz, CDCl₃) δ 7.33-7.16 (multiple signals, 35H+residual CHCl₃), 5.48 (dd, J_{F,H}=53.15 Hz, J_{H,H}=2.2 Hz, 1H), 5.50 (s, 1H), 4.88 (d, J=11.0 Hz, 1H), 4.72-4.63 (multiple signals, 2H), 4.62-4.43 (multiple signals, 12H), 4.27 (dd, J=9.6, 4.1 Hz, 1H), 4.03 (app t, J=9.5 Hz, 1H), 3.92 (dd, J=9.3, 3.1 Hz, 1H), 3.88-3.79 (multiple signals, 5H), 3.77-3.65 (multiple signals, 3H), 3.60 (app dt, J=16.8, 2-3 Hz, 1H). ¹³C NMR (100 MHz, CDCl₃) δ 138.5, 138.3, 138.2, 138.1, 138.0, 137.7, 137.3, 128.41 (2C), 128.38 (2C), 128.29 (2C), 128.26 (2C), 128.21 (2C), 128.19 (4C), 127.86 (2C), 127.85, 127.84, 127.79 (2C), 127.69 (2C), 127.67 (2C), 127.66 (4C), 127.56 (2C), 127.54, 127.52, 127.49, 127.4, 127.3, 106.1 (C—F, d, ¹J_{CF}=221.9 Hz), 82.1, 80.2, 77.6 (C—F, d, ³J_{CF}=3.32 Hz), 76.2, 74.9 (2C), 74.2, 73.31, 73.30, 73.1, (72.7, 72.5, 72.2, 71.9, 71.8, 6C with 2 overlapping signals), 69.4, 69.0, 46.79 (C—F, d, ²J_{CF}=22.10 Hz). ¹⁹F NMR (376 MHz, CDCl₃) δ −129.83 (dd, J=53, 16.8 Hz). [α]_{D}²⁵: +12.65 (c1.0, CHCl₃). IR (cm⁻¹): 3029, 2866, 2251, 1973, 1363, 1087. HRMS (ESI-TOF) m/z: [M+Na]⁺: Calcd. for C₆₁H₆₃FO₉SNa: 1013.4075, found 1013.4073.

### Example 2—Synthesis of S-Man₃ Oligosaccharide

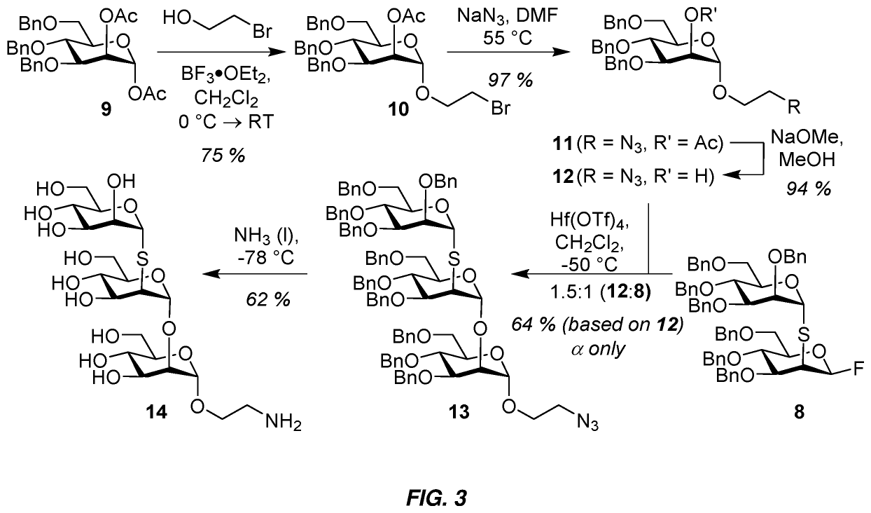

With dimannose donor 8 in hand, a suitable monomannose acceptor was prepared to produce Mani. Starting from mannose building block 9 (Norberg et al., “Synthesis and Binding Affinity Analysis of alpha1-2- and alpha1-6-O/S-linked Dimannosides for the Elucidation of Sulfur in Glycosidic Bonds Using Quartz Crystal Microbalance Sensors,” Carbohydr. Res. 452:35-4 (2017), which is hereby incorporated by reference in its entirety), installation of an azidoethyl linker and deprotection at the 2-position efficiently afforded acceptor 12. Glycosylation of 12 with 1.5 equivalents of 8, in the presence of hafnium trifluoromethanesulfonate (Manabe et al., “Hafnium(IV) Tetratriflate as a Glycosyl Fluoride Activation Reagent,” J. Org. Chem. 78:4568-4572 (2013), which is hereby incorporated by reference in its entirety) afforded the desired trisaccharide 13 in 64% yield as a single stereoisomer. After global deprotection with sodium in liquid ammonia the desired S-Man₃ 14 was isolated in 62% yield (FIG. 3). The stability of the thio linkage under dissolving metal conditions has been observed previously (Norberg et al., “Synthesis and Binding Affinity Analysis of alpha1-2- and alpha1-6-O/S-linked Dimannosides for the Elucidation of Sulfur in Glycosidic Bonds Using Quartz Crystal Microbalance Sensors,” Carbohydr. Res. 452:35-4 (2017); Zhong et al., “Probing the Substrate Specificity of Golgi alpha-mannosidase II by Use of Synthetic Oligosaccharides and a Catalytic Nucleophile Mutant,” J. Am. Chem. Soc. 130:8975-83 (2008), each of which is hereby incorporated by reference in its entirety), but is nevertheless noteworthy. The α configuration of all mannose units was confirmed by carbon-coupled HSQC, which showed all ¹J_{CH} to be in the range of 171-178 Hz (Crich et al., “Direct Stereoselective Synthesis of β-Thiomannosides,” J. Org. Chem. 65:801-805 (2000); Bock et al., “A Study of 13CH Coupling Constants in Hexopyranoses,” J. Chem. Soc, Perkin Trans. 2:293-297 (1974), each of which is hereby incorporated by reference in its entirety).

Individual reaction steps and evaluation by ¹H, ¹³C, ¹⁹F and/or HSQC NMR are described below.

### 2-Bromoethyl-2-O-acetyl-3,4,6-tri-O-benzyl-α-D-mannopyranoside (10)

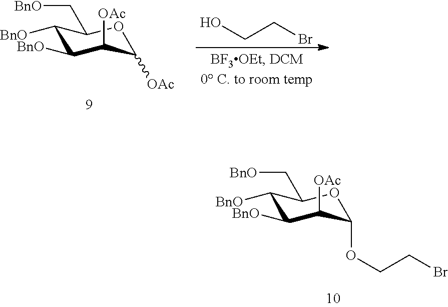

A solution of 1,2-di-O-acetyl-3,4,6-tri-O-benzyl-α/β-D-mannopyranose 9 (John, et al., “Synthesis of Truncated Analogues for Studying the Process of Glycosyl Phosphatidylinositol Modification,” Organic Letters 12:2080-2083 (2010), which is hereby incorporated by reference in its entirety) (1.1 g; 2.06 mmol; 1 equiv.) and bromoethanol (365 μL; 5.14 mmol; 2.5 equiv.) in dry dichloromethane (11 mL) was cooled to 0° C. and BF₃·OEt₂ (1.41 mL; 10.29 mmol; 5 equiv.) was added dropwise. The ice bath was allowed to melt and warm to room temperature as the stirred overnight. The reaction mixture was diluted with 40 mL of dichloromethane and washed with cold aq. sat. NaHCO₃ and brine solution. The organic layers were dried over MgSO₄, concentrated and crude was purified by column chromatography (ethyl acetate: hexane; 3:1) to afford compound 10 (925 mg; 1.54 mmol) as a syrup in 75% yield. ¹H NMR (400 MHz, CDCl₃) δ 7.35-7.21 (multiple signals, 13H+residual CHCl₃), 7.18-7.13 (m, 2H), 5.38 (dd, J=3.4, 1.8 Hz, 1H), 4.89 (d, J=1.8 Hz, 1H), 4.86 (d, J=10.8 Hz, 1H), 4.71 (d, J=11.2 Hz, 1H), 4.67 (d, J=12.4 Hz, 1H), 4.55 (d, J=11.2 Hz, 1H), 4.51 (d, J=12.4 Hz, 1H), 4.47 (d, J=10.8 Hz, 1H), 4.02-3.91 (m, 2H), 3.91-3.84 (m, 2H), 3.84-3.74 (m, 2H), 3.71 (d, J=10.7 Hz, 1H), 3.47 (t, J=6.1 Hz, 2H), 2.15 (s, 3H). ¹³C NMR (100 MHz, CDCl₃) δ (carbonyl C not observed), 138.3, 138.1, 137.9, 128.4 (2C), 128.3 (4C), 128.1 (2C), 127.9 (2C), 127.74 (2C), 127.73 127.63, 127.58, 98.0, 78.1, 75.2, 74.2, 73.4, 71.9, 71.8, 68.8, 68.6, 67.9, 29.9, 21.1. [α]_{D}²⁵: +3.2 (c1.0, CHCl₃). IR (cm⁻¹): 2915, 2015, 1745, 1237, 1095. HRMS (ESI-TOF) m/z: [M+Na]⁺: Calcd. for C₃₁H₃₅BrO₇Na: 621.1464, found 621.1453.

### 2-azidoethyl-2-O-acetyl-3,4,6-tri-O-benzyl-α-D-mannopyranoside (11)

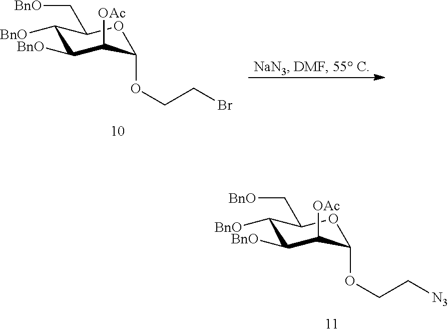

A solution of the compound 10 (910 mg; 1.52 mmol; 1 equiv.) and sodium azide (493 mg; 7.59 mmol; 5 equiv.) in dry DMF (25 mL) was stirred at 55° C. for 6 hours. After completion of the reaction, solvent was co-evaporated with toluene under reduced pressure, and the residue was dissolved in the mixture of water and ethyl acetate and extracted with ethyl acetate. The organic layers were dried over MgSO₄, concentrated and residue was purified by column chromatography (ethyl acetate and hexane; 2:1) to afford compound 11 as colorless syrup (826 mg; 1.47 mmol) in 97% yield.

¹H NMR (400 MHz, CDCl₃) δ 7.30 (multiple signals+residual CHCl₃, 13H), 7.15 (dd, J=7.3, 2.2 Hz, 2H), 5.39 (app t, J=1.9 Hz, 1H), 4.89 (d, J=1.8 Hz, 1H), 4.86 (d, J=10.8 Hz, 1H), 4.68 (dd, J=11.6, 9.6 Hz, 2H), 4.01 (dd, J=8.9, 3.3 Hz, 1H), 3.94-3.75 (m, 4H), 3.71 (dd, J=10.4, 1.5 Hz, 1H), 3.65-3.57 (m, 1H), 3.44-3.31 (m, 2H), 2.14 (s, 3H). ¹³C NMR (100 MHz, CDCl₃) δ 170.4, 138.3, 138.1, 137.8, 128.4, 128.3, 128.3, 128.1, 127.8, 127.7, 98.0, 78.0, 75.1, 74.1, 73.4, 71.9, 71.7, 68.8, 68.6, 66.7, 50.4, 21.1. [α]_{D}²⁵: +1.9 (c1.0, CHCl₃). IR (cm⁻¹): 3030, 2866, 2103, 1743, 1233, 1088. HRMS (ESI-TOF) m/z: [M+Na]⁺: Calcd. for C₃₁H₃₅N₃O₇Na: 584.2373, found 584.2372.

### 2-azidoethyl-3,4,6-tri-O-benzyl-α-D-mannopyranoside (12)

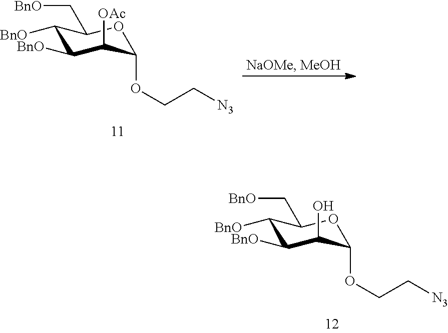

Compound 11 (810 mg; 1.44 mmol; 1 equiv.) was dissolved in methanol (4 mL) and dichloromethane (4 mL) and 65 μL (289 μmol; 0.2 equiv.) 25% sodium methoxide in methanol solution was added and stirred reaction mixture for 2 hours at room temperature. Reaction was neutralized with Amberlite IR-120 H⁺ form ion exchange resin. Resin was filtered off and the reaction mixture was concentrated under reduced pressure. The residue was purified by column chromatography to afford compound 12 (704 mg; 1.36 mmol) as colorless syrup in 94% yield.

¹H NMR (400 MHz, CDCl₃) δ 7.35-7.23 (multiple signals, 13H+residual CHCl₃), 7.21-7.14 (m, 2H), 4.94 (d, J=1.7 Hz, 1H), 4.82 (d, J=10.8 Hz, 1H), 4.68 (app s, 2H), 4.63 (d, J=12.2 Hz, 1H), 4.52 (d, J=12.2 Hz, 1H), 4.50 (d, J=10.8 Hz, 1H), 4.05 (br s, 1H), 3.92-3.66 (multiple signals, 6H), 3.61 (ddd, J=10.6, 6.7, 3.7 Hz, 1H), 3.44-3.27 (m, 2H), 2.54 (s, 1H). ¹³C NMR (100 MHz, CDCl₃) δ 138.3, 138.2, 137.8, 128.5, 128.4, 128.3, 127.95, 127.93, 127.92, 127.8, 127.7, 127.6, 99.4, 80.0, 75.1, 74.1, 73.5, 72.1, 71.4, 68.9, 68.1, 66.6, 50.5. [α]_{D}²⁵: +22.3 (c1.0, CHCl₃). IR (cm⁻¹): 3436, 3030, 2914, 2101, 1960, 1495, 1060. HRMS (ESI-TOF) m/z: [M+Na]⁺: Calcd. for C₂₉H₃₃N₃O₇Na: 542.2267, found 542.2265.

### 2-azidoethyl-3,4,6-tri-O-benzyl-2-O-[3,4,6-tri-O-benzyl-2-S-α-D-mannopyranosyl-(1→2)]-α-D-mannopyranoside (13)

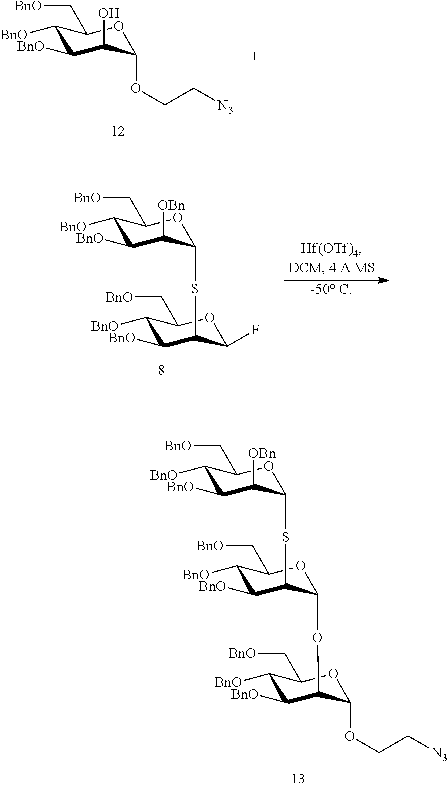

Disaccharide donor 8 (300 mg; 303 μmol; 1 equiv.) and acceptor 12 (235 mg; 454 μmol; 1.5 equiv.) were dissolved in of anhydrous dichloromethane (3 mL), and freshly crushed and flame dried 4 Å molecular sieves were added and stirred for 15 min at room temperature. The mixture was cooled at −50° C., then Hf(OTf)₄ (234 mg; 303 μmol; 1 equiv.) was added and allowed to stir for next 1 h. The reaction was neutralized with a few drops of triethylamine, diluted with CH₂Cl₂, filtered through a celite pad and concentrated. Purification by column chromatography (EtOAc:Hexane, 1:5) afforded trisaccharide 13 as white foam in 64% yield (287 mg, 194 μmol).

¹H NMR (400 MHz, CDCl₃) δ 7.40-7.08 (multiple signals, 50H+residual CHCl₃), 5.59 (s, 1H), 5.33 (s, 1H), 4.97 (d, J=1.8 Hz, 1H), 4.94-4.80 (multiple signals, 3H), 4.71-4.45 (multiple signals, 15H), 4.40-4.32 (multiple signals, 2H), 4.23 (dd, J=9.3, 4.3 Hz, 1H), 4.18-4.10 (m, 1H), 4.09-4.02 (m, 2H), 4.01-3.95 (m, 1H), 3.94-3.89 (m, 2H), 3.88-3.64 (multiple signals, 11H), 3.59 (dd, J=11.0, 1.7 Hz, 1H), 3.32 (ddd, J=10.5, 6.0, 4.1 Hz, 1H), 3.18 (m, 2H). ¹³C NMR (100 MHz, CDCl₃) δ 138.7, 138.6, 138.50, 138.47, 138.45, 138.39, 138.37, 138.3, 138.1 (two resonances), 128.5, 128.4, 128.38 (multiple resonances), 128.37, 128.35, 128.30 (multiple resonances), 128.26, 128.00, 127.95, 127.93, 127.92, 127.90, 127.83, 127.79, 127.77, 127.72, 127.68, 127.67, 127.66, 127.63, 127.57, 127.55, 127.54, 127.50, 127.44, 127.41, 102.7, 98.9, 83.1, 80.5, 79.4, 78.9, 75.8, 75.7, 75.17, 75.14, 74.96, 74.90, 74.7, 73.5, 73.3, 73.1, 72.45, 72.40, 72.1, 72.00, 71.98, 71.4, 69.6, 69.3, 69.0, 66.4, 50.4, 49.5. [α]_{D}²⁵: +5.1 (c1.0, CHCl₃). IR (cm⁻¹): 3035, 2358, 2167, 1981, 1219; HRMS (ESI-TOF) m/z: [M+Na]⁺: Calcd. for C₉₀H₉₅N₃O₁₅SNa: 1512.6382, found 1512.6356.

### 2-aminoethyl-[α-D-mannopyranosyl-(1→2)-2-S-{α-D-mannopyranosyl-(1→2)}]-α-D-mannopyranoside (14)

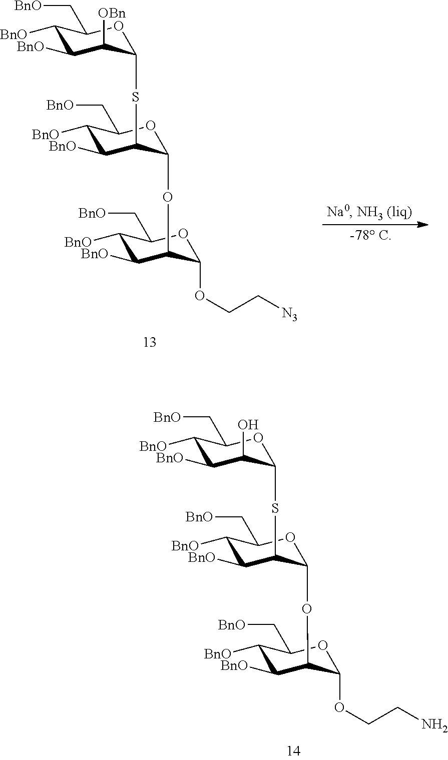

Into an oven dried 50 mL 3-necked flask, ˜10 mL ammonia was condensed under stream of nitrogen at −78° C. Next, 23 mg of (1.0 mmol, 100 equiv.) Na^{o} was added, and the bright blue reaction was allowed to stir for 1 hour to ensure that the color stably persisted. 15 mg (10 μmol; 1 equiv.) of trisaccharide 13 in 1 mL of dry THF was added by syringe, and the reaction was allowed to stir for 4 hours. When reaction was finished (monitored by direct-infusion LC-MS), solid NH₄Cl was added portionwise until the disappearance of blue color and the reaction mixture was warmed to room temperature. The crude product was desalted on a Biogel P-2 size exclusion gel column followed by lyophilization, affording 3.5 mg (6.2 μmol) of deprotected trisaccharide 14 as a glassy white solid in 62% yield.

¹H NMR (400 MHz, D₂O) δ 5.41 (s, 1H), 5.27 (s, 1H), 5.12 (s, 1H), 4.26 (dd, J=9.6, 4.6 Hz, 1H), 4.12 (dd, J=3.4, 1.5 Hz, 1H), 4.06-3.59 (m, 17H), 3.51 (t, J=9.7 Hz, 1H), 3.33-3.20 (m, 2H). ¹³C NMR (100 MHz, D₂O) δ 103.0, 98.1, 86.8, 78.4, 73.4 (multiple resonances), 72.9, 71.3, 70.8, 69.9, 69.0, 67.8, 67.0, 66.8, 63.7, 60.8 (multiple resonances), 52.2, 39.0. [α]_{D}²⁵: −1133.9 (c1.0, H₂O) IR (cm⁻¹): 3422(b), 3016, 2360, 2051, 1625, 1437; HRMS (ESI-TOF) m/z: [M+Na]⁺: Calcd. for C₂₀H₃₇NO₁₅SNa: 586.1782, found 586.1770.

### Example 3—Synthesis of S-Man₄ Oligosaccharide

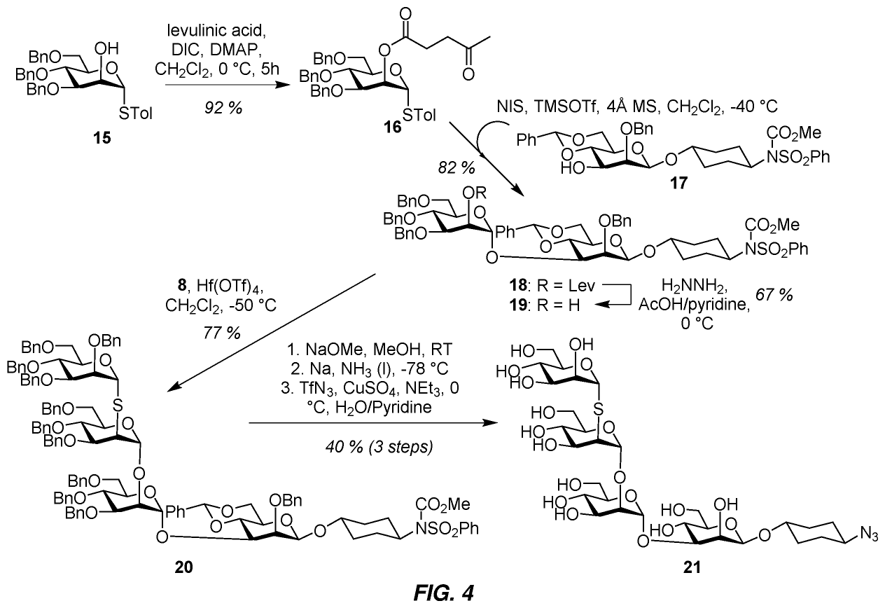

Similarly, preparation of an S-Man₄ was carried out. This oligosaccharide contains a reducing-terminal β-mannose analogous to the core mannose in the natural Man₉GlcNAc₂. Dimannose acceptor 19 was prepared by coupling the previously-described β-mannose core 17 (MacPherson et al., “Multivalent Glycocluster Design through Directed Evolution,” Angew. Chem. Int. Ed. 50:11238-11242 (2011), which is hereby incorporated by reference in its entirety) to known building block 16 (Chayajarus et al., “Stereospecific Synthesis of 1,2-cis Glycosides by Vinyl-Mediated IAD,” Org. Lett. 6:3797-800 (2004), which is hereby incorporated by reference in its entirety), followed by Lev deprotection. Acceptor 19 coupled smoothly to Man₂ fluoride donor 8 (see FIG. 4) in 77% yield, again as single stereoisomer. This tetrasaccharide was globally deprotected and converted to azide 21 in three steps with an overall yield of 40%. 21 exhibited three anomeric ¹J_{CH} values from 169-174 Hertz for the α linkages, and, as expected, a value of 158 Hz for β linkage.

Individual reaction steps and evaluation by ¹H, ¹³C, ¹⁹F and/or HSQC NMR are described below.

### p-Tolyl-3,4,6-tri-O-benzyl-2-O-(4-oxopentanoyl)-1-thio-α-D-mannopyranoside (16)

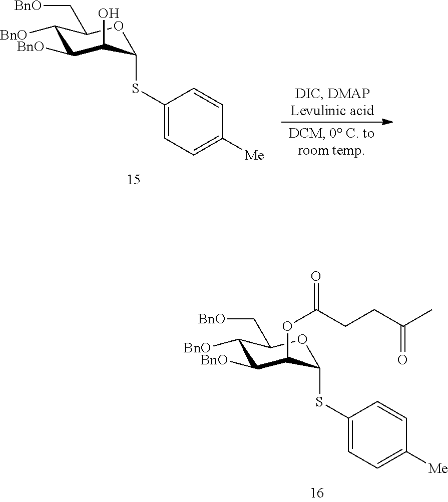

To a solution of alcohol 15 (Alam et al., “Mimicry of an HIV Broadly Neutralizing Antibody Epitope with a Synthetic Glycopeptide,” Sci Transl Med 9 (2017), which is hereby incorporated by reference in its entirety) (600 mg; 1.077 mmol; 1 equiv.) in anhydrous CH₂Cl₂ (10 mL), DMAP (26 mg; 0.215 mmol; 0.2 equiv.), levulinic acid (0.21 mL; 2.1 mmol; 2 equiv.) and N,N′-diisopropylcarbodiimide (0.33 mL; 2.1 mmol; 2 equiv.) were added sequentially at 0° C. The reaction mixture was gradually warmed up to ambient temperature and stirred for 5 hours. After complete consumption of the starting alcohol, the reaction was concentrated in vacuo and purified by flash chromatography (30% ethyl acetate in hexane) to afford corresponding levulinoate ester 16 (648 mg; 0.99 mmol; 92%) as white foam.

¹H NMR (400 MHz, CDCl₃) δ 7.42-7.22 (multiple signals, 15H+residual CHCl₃), 7.20 (m, 2H), 7.04 (d, J=7.9 Hz, 2H), 5.58 (dd, J=2.8, 1.7 Hz, 1H), 5.44 (d, J=1.7 Hz, 1H), 4.88 (d, J=10.8 Hz, 1H), 4.70 (d, J=11.2 Hz, 1H), 4.63 (d, J=11.9 Hz, 1H), 4.54 (d, J=11.2 Hz, 1H), 4.52 (d, J=10.8 Hz, 1H), 4.46 (d, J=11.9 Hz, 1H), 4.34 (m, 1H), 3.94 (dd, J=10.7, 2.8 Hz, 1H), 3.91 (app t, 9 Hz, 1H), 3.84 (dd, J=10.9, 4.6 Hz, 1H), 3.71 (dd, J=10.9, 1.9 Hz, 1H), 2.82-2.62 (m, 4H), 2.29 (s, 3H), 2.11 (s, 3H). ¹³C NMR (100 MHz, CDCl₃) δ 206.2, 171.9, 138.3, 138.2, 137.9, 137.7, 132.4, 129.8, 129.7, 128.4, 128.3, 128.3, 128.1, 127.9, 127.8, 127.7, 127.7, 127.5, 86.4, 78.4, 75.2, 74.5, 73.3, 72.3, 71.7, 70.4, 68.9, 37.9, 29.7, 28.1, 21.1. [α]_{D}²⁵: +99.4 (c1.0, CHCl₃). IR (cm⁻¹): 2908, 2861, 2660, 1739, 1718, 1101. HRMS (ESI-TOF) m/z: [M+Na]⁺: Calcd. for C₃₉H₄₂O₇SNa: 677.2549, found: 677.2542.

### 2-O-benzyl-3-O-(3,4,6-tri-O-benzyl-2-O-(4-oxopentanoyl)-α-D-mannopyranosyl)-4,6-O-benzylidine-β-D-mannopyranoside (18)

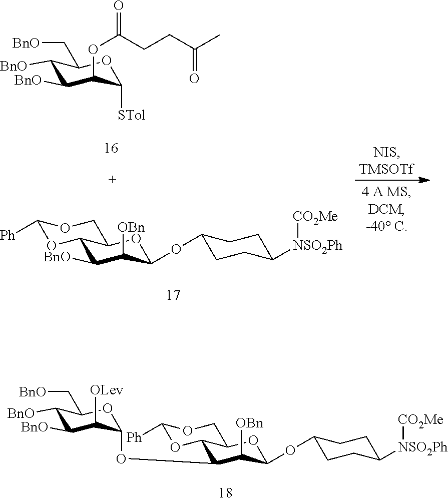

500 mg (0.763 mmol; 1 equiv.) of 16 and 598 mg (0.915 mmol; 1.2 equiv.) of 17⁶ in a 50 mL flask were dissolved in toluene and cooled to −78° C. Vacuum was applied and the cooling bath was removed and allowed to warm to room temperature as the toluene evaporated. This procedure was repeated twice. The dry residue was dissolved in 14 mL of CH₂Cl₂ and activated 4 Å molecular sieves were added stirred for 20 minutes at room temperature. The reaction mixture was cooled to −40° C. and stirred another 15 minutes more. 429 mg of NIS (1.91 mmol; 2.5 equiv.) was added, and after 15 minutes, 27 μL of TMSOTf (0.15 mmol; 0.2 equiv.) dissolved in 2 mL of CH₂Cl₂ was added dropwise. The reaction was stirred at −40° C. for 2 h and quenched with aq. sat. NaHCO₃ and solid Na₂S₂O₃. The reaction mixture was filtered, and the filtrate was washed with aq. sat. Na₂S₂O₃, aq. sat. NaHCO₃ and brine, then the organic layer was dried (MgSO₄) and concentrated prior to flash column chromatography (25% ethyl acetate and hexane). Compound 18 (741 mg; 0.626 mmol; 82%) was obtained as a white amorphous solid.

¹H NMR (400 MHz, CDCl₃) δ 7.92 (app d, J=8 Hz, 2H), 7.62 (app t, J=7.2 Hz, 1H), 7.53 (app t, J=8 Hz, 2H), 7.5 (m, 2H), 7.41-7.10 (multiple signals, 23H), 5.60 (s, 1H), 5.58 (dd, J=2.8, 1.3 Hz,1H), 5.23 (d, J=1.3 Hz, 1H), 4.92-4.83 (m, 2H), 4.78 (d, J=12.3 Hz, 1H), 4.66 (d, J=11.3 Hz, 1H), 4.57-4.38 (multiple signals, 6H), 4.32 (dd, J=10.4, 4.9 Hz, 1H), 4.18 (app t, J=9.7 Hz, 1H), 3.90 (multiple signals, 3H), 3.82-3.54 (multiple signals, 6H), 3.65 (s, 3H), 3.31 (app td, J=9.7, 4.9 Hz, 1H), 2.73-2.58 (m, 4H), 2.37-2.13 (m, 3H), 2.10 (s, 3H), 2.0-1.91 (m, 1H), 1.89-1.79 (m, 2H), 1.59-1.48 (m, 1H), 1.40-1.28 (m, 1H). ¹³C NMR (100 MHz, CDCl₃) δ 206.1, 202.2 171.7, 152.6, 140.3, 138.6, 138.3, 138.1, 137.9, 137.3, 133.4, 128.8, 128.7, 128.36 (two resonances), 128.34, 128.3, 128.2, 128.13, 128.10, 127.9, 127.75, 127.71, 127.67, 127.63, 127.61, 127.56, 126.0, 101.2, 100.1, 98.7, 78.6, 77.81, 77.75, 76.4, 75.2, 75.0, 74.9, 74.3, 73.4, 72.0, 71.5, 69.2, 68.5, 68.4, 67.4, 58.5, 53.5, 38.1, 33.2, 31.5, 29.8, 28.46, 28.44, 28.2. [α]_{D}²⁵: −3.6 (c1.0, CHCl₃). IR (cm⁻¹):3030, 2924, 2861, 1962, 1732, 1496, 1356, 1088. HRMS (ESI-TOF) m/z: [M+Na]⁺: Calcd. for C₆₆H₇₃NO₁₇SNa: 1206.4497, found 1206.4469.

### 2-O-benzyl-3-O-(3,4,6-tri-O-benzyl-α-D-mannopyranosyl)-4,6-O-benzylidine-β-D-mannopyranoside (19)

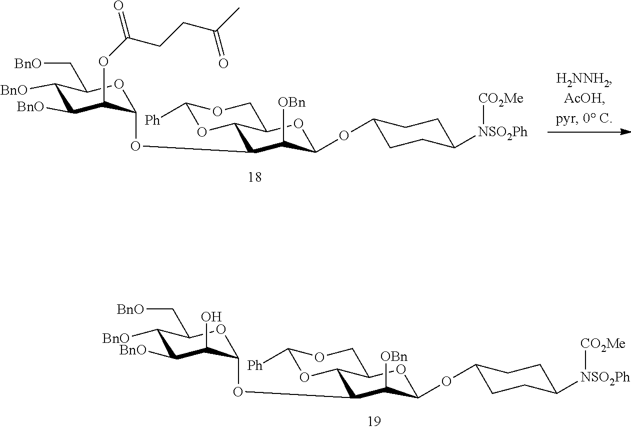

Compound 18 (700 mg; 0.591 mmol; 1 equiv.) was dissolved in 10 mL of CH₂Cl₂ and cooled to 0° C. Pyridine (190 μL; 2.364 mmol; 4 equiv.) and acetic acid (34 μL; 0.591 mmol; 1 equiv.) was mixed in a 1 mL ice cooled vial and added to reaction mixture dropwise, followed by addition of hydrazine (91 μL; 2.955 mmol; 5 equiv.) to reaction mixture. The reaction was stirred for 2 h at 0° C. and quenched with 2 mL of acetone. The reaction mixture was concentrated under reduced pressure and the crude was purified by column chromatography in 30% ethyl acetate and hexane to afford compound 19 (430 mg; 0.395 mmol; 67%) as white glassy solid.

¹H NMR (400 MHz, CDCl₃) δ 7.92 (app d, J=8 Hz, 2H), 7.62 (app t, J=7.4 Hz, 1H), 7.53 (app t, J=7.8 Hz, 2H), 7.47-7.12 (multiple signals, 25H+residual CHCl₃), 5.56 (s, 1H), 5.33 (d, J=1.7 Hz, 1H), 4.91 (d, J=12.4 Hz, 1H), 4.85 (d, J=11.1 Hz, 1H), 4.80 (d, J=12.4 Hz, 1H), 4.67 (d, J=11.4 Hz, 1H), 4.61 (d, J=11.4 Hz, 1H), 4.54-4.46 (m, 4H), 4.41 (tt, J=12.1, 3.8 Hz, 1H), 4.30 (dd, J=10.4, 4.8 Hz, 1H), 4.18 (dd, J=2.8, 1.7 Hz, 1H), 4.13 (app t, J=9.7 Hz, 1H), 4.00 (dd, J=10.1, 3.1 Hz, 1H), 3.91 (app t, J=10.3 Hz, 1H), 3.87-3.80 (m, 2H), 3.79-3.52 (m, 5H), 3.66 (s, 3H), 3.33 (app td, J=9.7, 4.9 Hz, 1H), 2.40 (br s, 1H), 2.35-2.10 (m, 3H), 2.00-1.9 (m, 1H), 1.9-1.8 (m, 2H), 1.59-1.45 (m, 1H+H₂O), 1.39-1.26 (m, 1H). ¹³C NMR (101 MHz, CDCl₃) δ 152.5, 140.3, 138.5, 138.33, 138.28, 137.8, 137.4, 133.4, 128.95, 128.8, 128.5, 128.4, 128.33, 128.30, 128.28, 128.2, 127.94, 127.90, 127.88, 127.72, 127.71, 127.59, 127.58, 127.56, 126.0, 101.5, 100.3, 100.1, 79.9, 78.7, 78.1, 76.4, 75.3, 74.93, 74.90, 74.3, 73.4, 71.9, 71.8, 69.3, 68.6, 68.0, 67.4, 58.4, 53.5, 33.2, 31.5, 30.9, 28.4. [α]_{D}²⁵: −2.01 (c1.0, CHCl₃). IR (cm⁻¹): 3540, 2919, 1732, 1452, 1666, 1359, 1086. HRMS (ESI-TOF) m/z: [M+Na]⁺: Calcd. for C₆₁H₆₇NO₁₅SNa: 1108.4129, found 1108.4065.

### Trans-(N-methylcarbonate-N-1-phenylsulfonyl)-cyclohexyl-2-O-benzyl-4,6-O-benzylidine-3-O-[{3,4,6-tri-O-benzyl-α-D-mannopyranosyl-(1→2)}-2-O-{3,4,6-tri-O-benzyl-α-D-mannopyranosyl-(1→2)}-2-S-{3,4,6-tri-O-benzyl-α-D-mannopyranosyl-(1→2)}]-β-D-mannopyranoside (20)

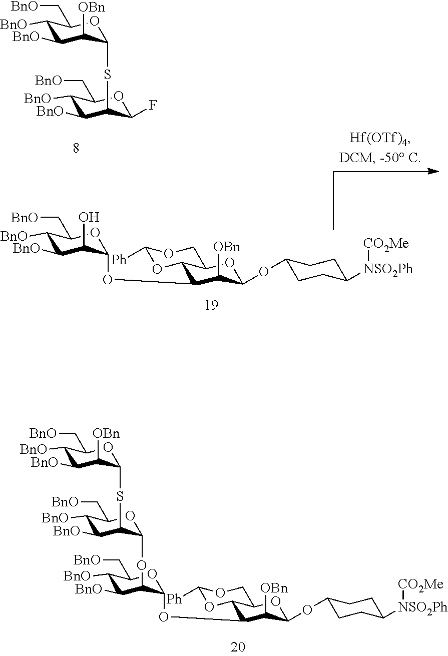

Disaccharide donor 8 (280 mg; 282 μmol; 1 equiv.) and acceptor 19 (230 mg; 212 μmol; 0.75 equiv.) were dissolved in anhydrous dichloromethane (5 mL), and freshly crushed and flame dried 4 Å molecular sieves was added and stirred for 15 min at room temperature. The mixture was cooled at −50° C., then Hg(OTf)₄ (219 mg; 282 μmol; 1 equiv.) was added and allowed to stir for 1 h. The reaction was neutralized with a few drops of triethylamine, diluted with CH₂Cl₂ and filtered through celite pad and concentrated, and then purification by column chromatography by 35% of EtOAc in Hexane, afforded trisaccharide 20 as white foam in 77% yield based on acceptor 19 (335 mg, 163.84 μmol).

¹H NMR (400 MHz, CDCl₃) δ 7.92 (app d, J=7.4 Hz, 2H), 7.61 (app t, J=7.4 Hz, 1H), 7.40 (t, J=7.7 Hz, 2H), 7.44-6.98 (multiple signals, 60H+residual CHCl₃), 5.57 (s, 1H), 5.36 (s, 2H), 5.33 (s, 1H), 5.01-4.73 (multiple signals, 5H), 4.62-4.30 (multiple signals, 17H), 4.29-4.15 (multiple signals, 5H), 4.08-3.97 (multiple signals, 3H), 3.96-3.39 (m, 20H), 3.30 (d, J=11.0 Hz, 1H), 3.12 (app td, J=9.6, 4.9 Hz, 1H), 2.34-2.16 (m, 2H), 2.16-2.08 (m, 1H), 2.00-1.69 (m, 3H), 1.57-1.43 (m, 1H), 1.34-1.19 (m, 1H). ¹³C NMR (100 MHz, CDCl₃) δ (selected signals) 152.5, 140.3, 138.9, 138.76, 138.63, 138.52, 138.50, 138.32, 138.28, 138.22 (two resonances), 138.1 (two resonances), 137.3, 133.4, 128.8, 128.46, 128.40, 128.38, 128.34 (multiple resonances), 128.31, 128.28 (multiple resonances), 128.23, 128.21, 128.17, 128.12, 128.0, 128.0, 127.9, 127.8, 127.75, 127.71, 127.66, 127.64, 127.59, 127.54, 127.51, 127.49, 127.47, 127.40, 127.3, 127.1, 125.95, 102.1, 101.3, 99.9, 99.8, 83.0, 80.4, 78.9, 78.3, 78.1, 76.3, 75.7, 75.5, 75.19, 75.14, 75.0, 74.9, 74.8, 74.0, 73.2, 73.1, 72.9, 72.5, 72.41, 72.46, 72.2, 71.9, 71.3, 69.7, 68.9, 68.8, 68.5, 67.3, 58.5, 53.4, 49.4, 33.1, 31.4, 28.4. [α]_{D}²⁵: +12.0 (c1.0, CHCl₃). IR (cm−¹): 3030, 2853, 1732, 1495, 1260. HRMS (ESI-TOF) m/z: [M+Na]⁺: Calcd. for C₁₂₂H₁₂₉NO₂₅S₂Na: 2078.8244, found 2078.8289.

### Trans-(N-1-phenylsulfonyl)-cyclohexyl-2-O-benzyl-4,6-O-benzylidine-3-O-[{3,4,6-tri-O-benzyl-α-D-mannopyranosyl-(1→2)}-2-O-{3,4,6-tri-O-benzyl-α-D-mannopyranosyl-(1→2)}-2-S-{3,4,6-tri-O-benzyl-α-D-mannopyranosyl-(1→2)}]-β-D-mannopyranoside (S3)

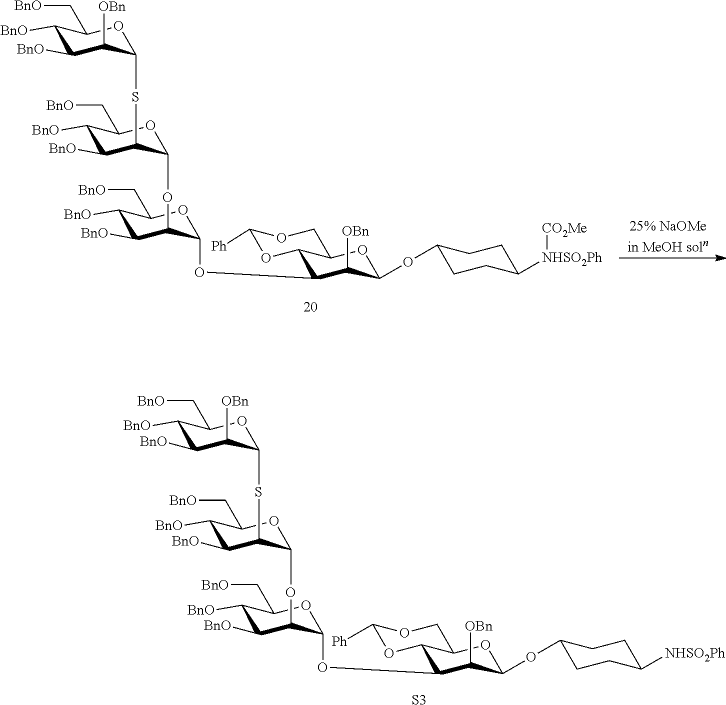

Compound 20 (280 mg; 136 μmol; 1 equiv.) was dissolved in methanol (2 mL) and dichloromethane (2 mL) and 10 μL (40.8 μmol; 0.3 equiv.) 25% sodium methoxide in methanol solution was added. After 3 hours at room temperature, the reaction was neutralized with Amberlite IR-120 H⁺ form ion exchange resin with caution to avoid acidifying below pH 6. The resin was filtered off and the filtrate was concentrated under reduced pressure. The concentrate was purified by column chromatography to afford compound S3 (244 mg; 122 μmol) as colorless syrup in 90% yield.

¹H NMR (400 MHz, CDCl₃) δ 7.88 (app d, J=7.1 Hz, 2H), 7.59 (app t, J=7 Hz, 1H), 7.52 (app t, J=7 Hz, 2H), 7.47-7.09 (multiple signals, 58H+residual CHCl₃), 7.08-6.98 (m, 2H), 5.56 (s, 1H), 5.33 (app s, 2H), 5.31 (s, 1H), 4.95-4.77 (multiple signals, 4H), 4.71 (d, J=12.3 Hz, 1H), 4.62-4.39 (multiple signals, 13H), 4.39-4.29 (multiple signals, 4H), 4.27-4.11 (multiple signals, 5H), 4.08-3.97 (m, 3H), 3.93-3.54 (multiple signals, 14H), 3.50-3.36 (m, 3H), 3.28 (d, J=10.6 Hz, 1H), 3.20-3.00 (m, 2H), 2.25-1.62 (m, 4H), 1.41-1.13 (m, 4H). ¹³C NMR (100 MHz, CDCl₃) δ (selected signals) 141.1, 139.0, 138.8, 138.7, 138.56, 138.54, 138.36, 138.34, 138.25, 138.23, 138.16, 138.13, 137.3, 132.7, 129.2, 129.0 128.48, 128.43, 128.41, 128.35, 128.33, 128.31, 128.29, 128.25, 128.22, 128.19, 128.12, 128.05, 127.9, 127.8, 127.75, 127.74, 127.71, 127.69, 127.61, 127.59, 127.57, 127.50, 127.45, 127.40, 127.3, 127.2, 126.9, 126.0, 101.3, 99.84, 99.82, 83.0, 80.5, 78.9, 78.3, 78.1, 75.8, 75.7, 75.5, 75.2, 75.1, 75.0, 74.95, 74.85, 74.0, 73.2, 73.15, 73.0, 72.54, 72.47, 72.37, 72.30, 71.9, 71.3, 69.7, 69.0, 68.9, 68.5, 67.4, 51.7, 49.4, 31.0 (multiple signals), 29.3. [α]_{D}²⁵: +8.0 (c1.0, CHCl₃). IR (cm⁻¹): 3029, 2862, 1731, 1496, 1452, 1090. HRMS (ESI-TOF) m/z: [M+Na]⁺: Calcd. for C₁₂₀H₁₂₇NO₂₅S₂Na: 2020.8189, found 2020.8182.

### Trans-aminocyclohexyl-3-O-[{α-D-mannopyranosyl-(1→2)}-2-O-{α-D-mannopyranosyl-(1→2)}-2-S-{α-D-mannopyranosyl-(1→2)}]-β-D-mannopyranoside (S4)

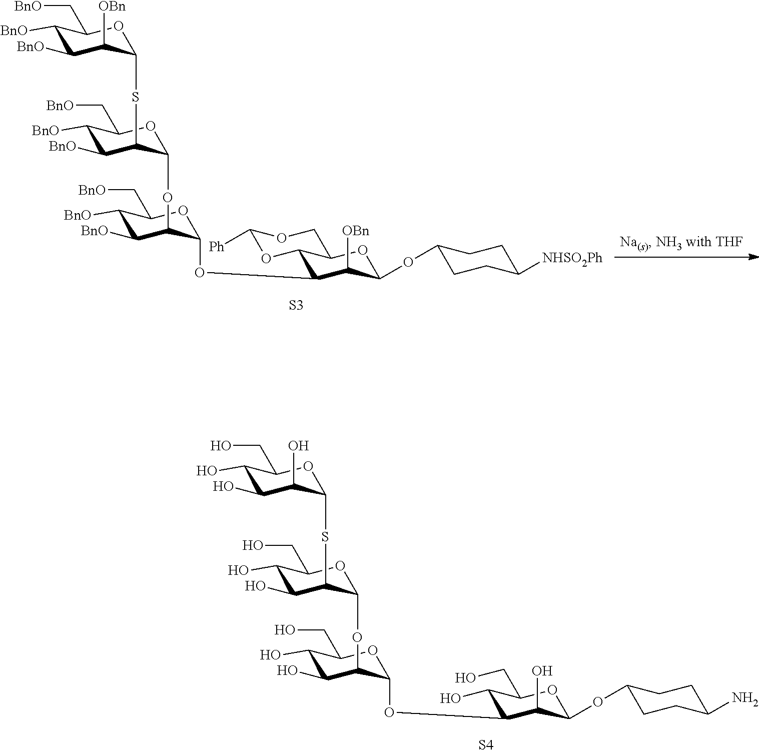

Into an oven dried 500 mL 3-necked flask, ˜200 mL ammonia was condensed under stream of nitrogen at −78° C. 471 mg of (20.5 mmol) Na^{o} was added, and the bright blue reaction was allowed to stir for 1 hour to observe persistence of the blue color. Next, 205 mg (103 μmol; 1 equiv.) of tetrasaccharide S3 in 2 mL of dry THF was added by syringe, and the reaction was allowed to stir for 2 hours. When reaction was finished (monitored by direct-infusion LCMS), solid NH₄Cl (548 mg; 10.3 mmol) was added portionwise until the disappearance of blue color and the ice bath was removed, allowing the mixture to warm to room temperature. The crude product was desalted on a Biogel P-2 size exclusion gel column and lyophilization afforded ˜105 mg of deprotected trisaccharide S4 (including some residual salt) as a hazy glassy white solid, which was used for the next step without further purification.

¹H NMR (400 MHz, D₂O) δ 5.13 (s, 1H), 5.06 (s, 1H), 5.00 (s, 1H), 4.54 (s, 1H), 3.99 (dd, J=9.7, 4.5 Hz, 1H), 3.84 (d, J=2.8 Hz, 1H), 3.81-3.77 (m, 2H), 3.76-3.68 (m, 2H), 3.68-3.33 (m, 17H), 3.28 (app t, J=9.5 Hz, 1H), 3.15-3.08 (m, 1H), 2.97-2.86 (m, 1H), 1.92-1.73 (m, 4H), 1.35-1.05 (m, 4H). ¹³C NMR (100 MHz, D₂O) δ 103.0, 100.4, 97.7, 86.7, 80.4, 78.4, 75.9, 73.4, 73.3, 73.2, 71.3, 70.8, 70.6, 69.9, 69.1, 67.5, 67.0, 66.8, 66.1, 60.82, 60.78, 60.6, 52.1, 49.0, 46.6, 30.2, 28.9, 28.1, 28.0, 8.1. HRMS (ESI-TOF) m/z: [M+Na]⁺: Calcd. for C₃₀H₅₃NO₂₀SNa: 802.2779, found 802.2761.

### Trans-azidocyclohexyl-3-O-[{α-D-mannopyranosyl-(1→2)}-2-O-{α-D-mannopyranosyl-(1→2)}-2-S-{α-D-mannopyranosyl-(1→2)}]-β-D-mannopyranoside (21)

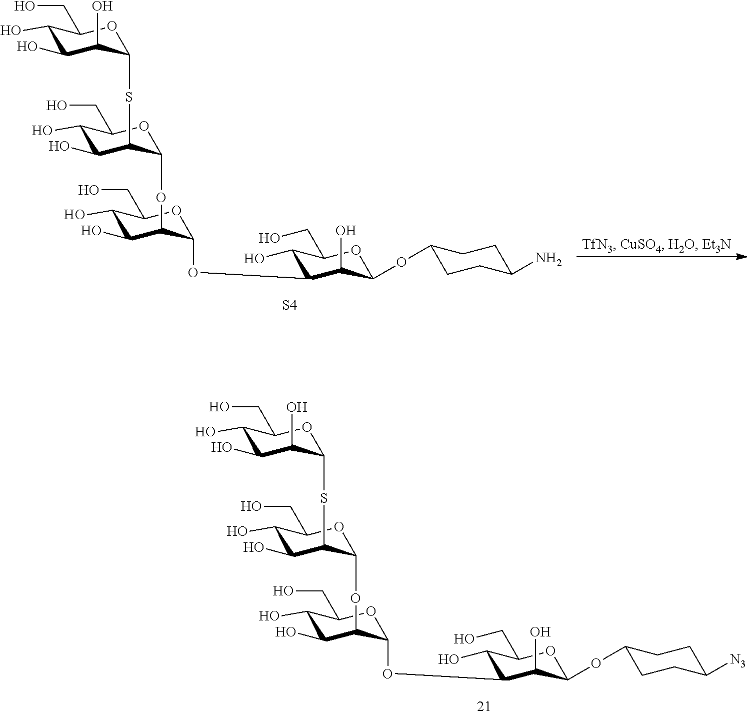

In a 5 ml oven-dried reaction vial with magnetic stir bar, 105 mg (1.62 mmol; 1 equiv.) of sodium azide was cooled to 0° C. and dissolved in 1.3 mL of pyridine, followed by treatment with 327 μL triflic anhydride (1.94 mmol; 1.2 equiv.). While this stirred for 2 hours at 0° C., in other 5 mL RB, ˜105 mg of crude tetrasaccharide amine S4 (used directly from previous step) was dissolved in 1.4 mL of water and cooled to 0° C. 5.1 mg of cupric sulphate (20.5 mmol; 0.2 equiv.) dissolved in 1 mL of H₂O was added to reaction mixture followed addition of 142 μL (143 mmol; 10 equiv.) of triethyl amine. The triflyl azide solution prepared above was added dropwise to tetrasaccharide reaction mixture at 0° C. and stirred for 1 hour. The crude material was desalted on a Biogel P-2 size exclusion gel column to yield 70 mg of crude product. The crude material was then purified by reverse phase HPLC (Column: Waters Xbridge Prep, C₁₈, 5 μm, 19×250 mm, 150 Å pore diameter. Method: 16 mL/min flow rate, A=H₂O; B=Acetonitrile without buffer; 1% B for 10 minutes, then 1-20% B over 55 minutes. Product was detected by UV at 220 nm. Product containing fractions from 3 injections were lyophilized to yield 33 mg (41.04 μmol) in 40% yield based on S3 as a colorless glass.

¹H NMR (800 MHz, D₂O, 293 K) δ 5.40 (s, 1H), 5.33 (s, 1H), 5.28 (s, 1H), 4.80 (s, 1H), 4.26 (dd, J=9.7, 4.6 Hz, 1H), 4.12 (dd, J=3.4, 1.5 Hz, 1H), 4.08-4.06 (m, 2H), 4.02-3.97 (m, 2H), 3.94-3.86 (m, 4H), 3.85-3.65 (m, 12H), 3.63 (dd, J=4.7, 1.5 Hz, 1H), 3.55 (app t, J=9.5 Hz, 1H), 3.53-3.48 (m, 1H), 3.39 (ddd, J=9.7, 6.2, 2.3 Hz, 1H), 2.12-1.94 (m, 4H), 1.52-1.30 (m, 4H). ¹³C NMR (200 MHz, D₂O) δ 103.1, 100.5, 97.7, 86.8, 80.5, 78.5, 76.0, 75.9, 73.41, 73.36, 73.26, 71.4, 70.9, 70.8, 70.0, 69.1, 67.6, 67.1, 66.9, 66.1, 60.9 (two resonances), 60.8, 60.6, 58.8, 52.2, 30.0, 28.6, 28.3, 28.1. [α]_{D}²⁵: −1533 (c1.0, H₂O). IR (cm⁻¹): 3370, 3266, 2236, 2173, 1970, 1063. HRMS (ESI-TOF) m/z: [M+Na]⁺: Calcd. for C₃₀H₅₁N₃O₂₀SNa: 828.2684, found 828.2676.

### Example 4—Evaluation of S-Man₃ and S-Man₄ Binding to HIV Broadly Neutralizing Antibody 2G12

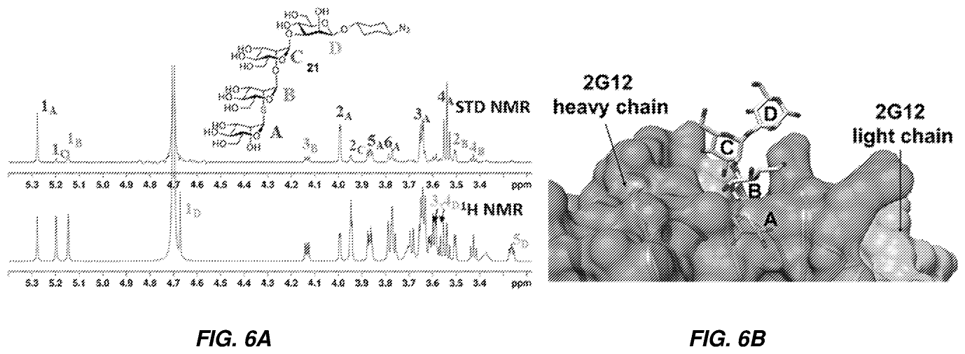

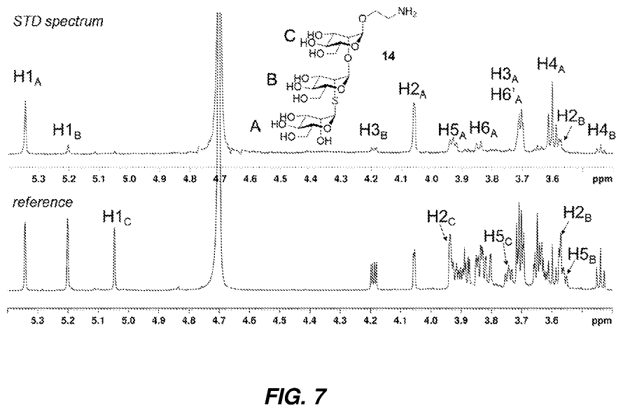

With these S-Man₃ and S-Man₄ derivatives in hand, their recognition by HIV broadly neutralizing antibody 2G12 was studied. 2G12 binds primarily to the linear trimannose (D1) arm of Man₉GlcNAc₂. STD-NMR (Saturation Transfer Difference NMR) spectroscopy with 25 μM 2G12 IgG and a 200:1 ratio of sugar:antibody in pH 6.7 deuterated phosphate buffered saline showed that, as expected, the greatest saturation transfer is seen for the non-reducing mannose unit in either Man₃ or Man₄ (FIGS. 6A, 7). The temperature was 298 K for S-Man₃ 14 and 293 K for S-Man₄ 21 in order to move the HDO peak to avoid covering an anomeric resonance. The experiments were performed without suppression of the residual HDO signal. On-resonance frequencies of 0.86 ppm δ (protein aliphatic) and 7 ppm δ (protein aromatic) were applied for S-Man₃ 14 and 293 K for S-Man₄ 21, respectively, in both cases with a saturation time of 3 s and an off-resonance frequency of 40 ppm δ.

In the case of the Man₄ derivative, negligible STD is observed for the reducing-terminal mannose unit. These data are closely analogous to STD NMR data previously acquired for oxygen-linked oligomannose fragments (Enriquez-Navas et al., “STD NMR Study of the Interactions Between Antibody 2G12 and Synthetic Oligomannosides That Mimic Selected Branches of gp120 Glycans,” ChemBioChem 13:1357-65 (2012); Enriquez-Navas et al., “A Solution NMR Study of the Interactions of Oligomannosides and the Anti-HIV-1 2G12 Antibody Reveals Distinct Binding Modes for Branched Ligands,” Chem. Eur. J. 17:1547-60 (2011), each of which is hereby incorporated by reference in its entirety), and are consistent with crystal structure data for Man₄ bound to 2G12, in which little if any interaction is evident between the antibody and residue D (FIG. 6B).

### Example 5—Analysis of Sulfur-Substituted Man₄ Derivatives Against the Action of Xanthomonas manihotis Mannosidase

Lastly, natural and sulfur-substituted Man₄ derivatives were tested against the action of Xanthomonas manihotis mannosidase, which cleaves oligomannose Manα1→2Man and Manα1→3Man linkages. S-Man₄ derivative 21 and its oxygen analog 22 were labeled by strain-promoted azide/alkyne cycloaddition (SPAAC) with DBCO amine linker 23, in order to facilitate separation and detection of degradation products by LC/MS. Man₄-cyclohexyl-N₃ 22 or the thioether derivative 21 were dissolved in degassed H₂O to afford a 25 mM stock, while the DBCO-NH₂ SPAAC reagent 23 was dissolved in acetonitrile (29 mM). Under a N₂ atmosphere, 1.1 equiv. of 23 was added to the Man₄ derivative, followed by heating to 37° C. and reaction monitoring by LC/MS. At 30 minutes, both SPAAC reactions were complete by LC/MS, showing two regioisomers, separable by LC/MS. Crude reactions were analyzed with an analytical C4 column (ACQUITY UPLC Protein BEH C4, 2.1 mm×150 mm, 300 Å, 1.7 μm) using the method in the Table 1 below with mobile phases A: H₂O (0.7% formic acid) and B: MeCN (0.1% formic acid).

TABLE 1
Column Parameters

| Time | Flow | | |
|---|---|---|---|
| (minutes) | (mL/min) | % A | % B |
| 0 | 0.3 | 90 | 10 |
| 1 | 0.3 | 90 | 10 |
| 7 | 0.3 | 70 | 30 |
| 7.05 | 0.3 | 5 | 95 |
| 10 | 0.3 | 5 | 95 |
| 10.05 | 0.3 | 90 | 10 |
| 13 | 0.3 | 90 | 10 |

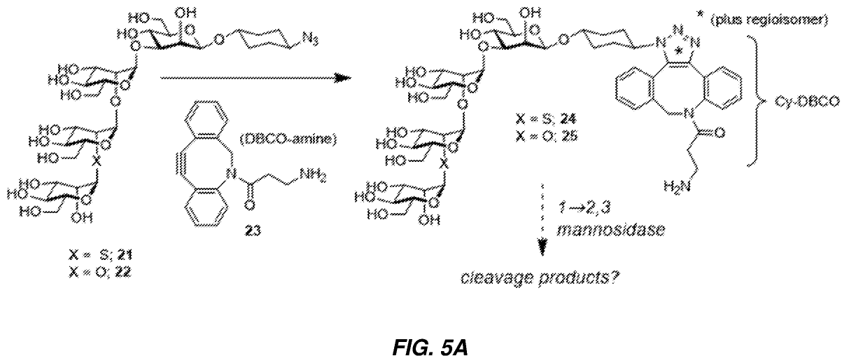

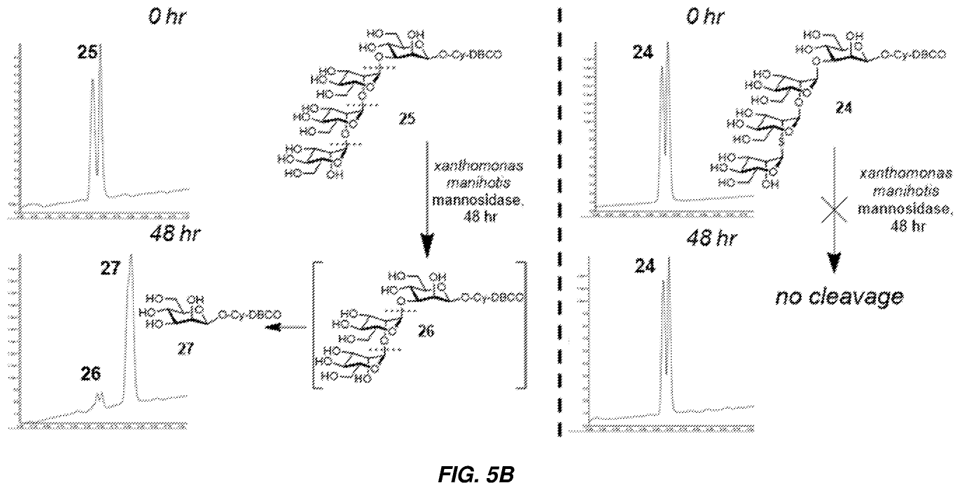

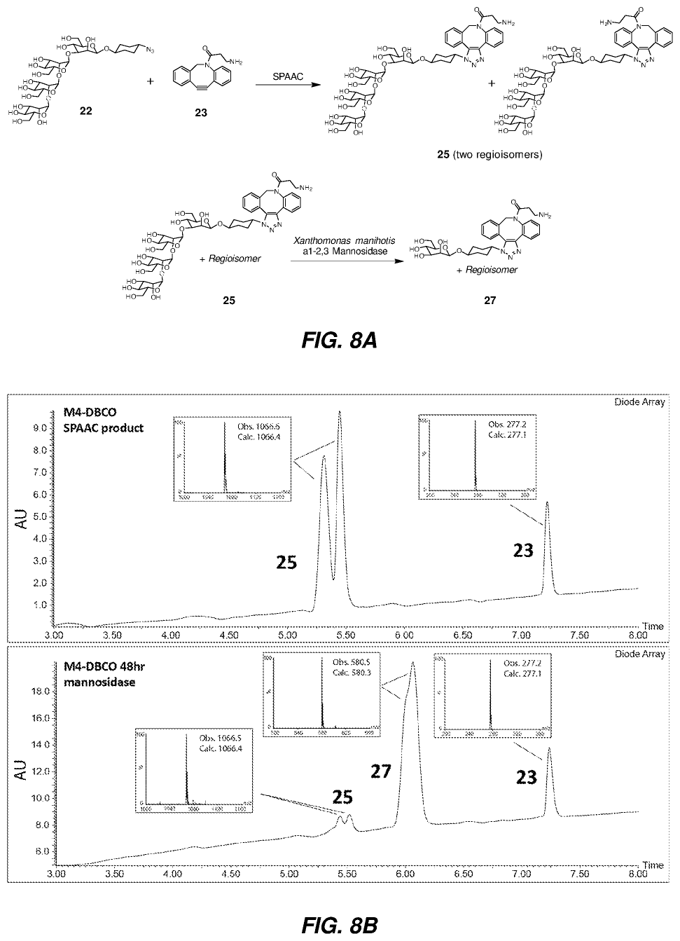

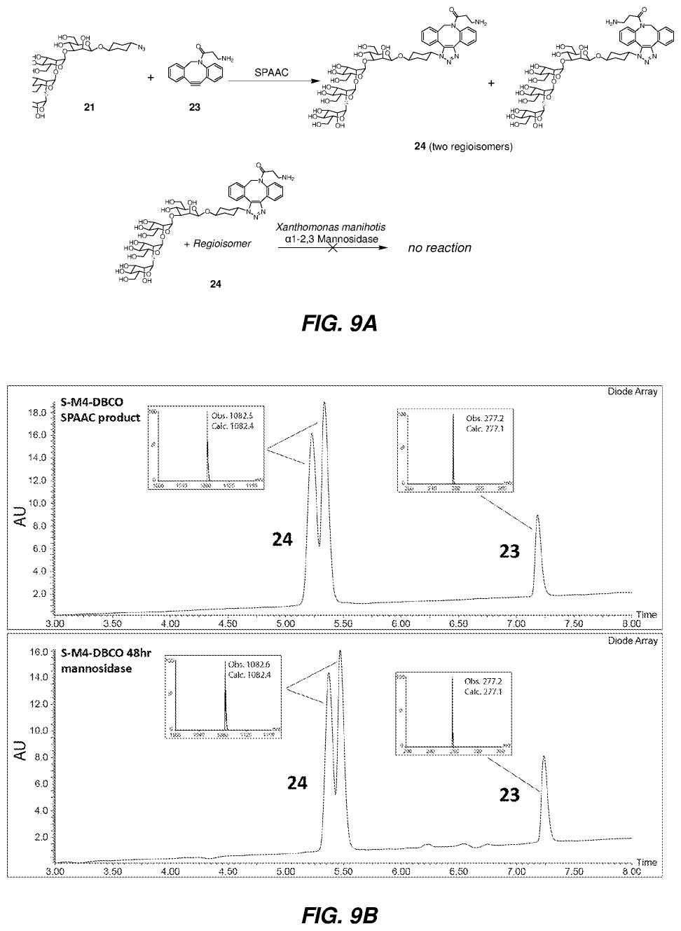

100 μg/mL DBCO-NH₂-labeled Man₄-derivative was digested with an α1-2,3 Mannosidase cloned from the pathogenic bacterium Xanthomonas manihotis (NEB: P0729S) according to manufacturer's instructions. Briefly, either Man₄-derivative was diluted into 1× glycobuffer I (provided with the enzyme) supplemented with 100 μg/mL BSA and finally 80 units of α1-2,3 Mannosidase. Reactions were monitored by LC/MS by the same method detailed above, over the course of 48 hr (see FIGS. 8A-B, 9A-B). After incubation with mannosidase, LC/MS analysis showed no degradation of sulfur-substituted derivative 24 after 48 hours, but nearly complete digestion of natural Man₄ derivative 25 to Man₁ 27 (FIGS. 5A-5B).

### Discussion

Based on the demonstrated results in Examples 1-5, a facile synthetic route to oligomannose derivatives with a non-reducing-terminal sulfur linkage that is highly resistant to enzymatic degradation. The S-Man₃ and S-Man₄ derivatives are recognized by a neutralizing HIV antibody, 2G12, through contacts that are similar to those it makes with the natural oligomannose structure. This synthetic strategy should be readily amenable to preparation of higher branched stabilized oligomannose analogs, such as thio-ether linked Man₉ and Man₉GlcNAc₂, which are suitable for immunogenicity studies. It is generally applicable to the synthesis of S-linked analogs of any glycan structure containing one or more non-reducing-terminal Man1α→2Man moieties. For example, Candida albicans glycans contain many branches terminating in this linkage, and it is similarly present in R. radiobacter Rv3 glycans (Gómez-Gaviria et al., “Role of Protein Glycosylation in Interactions of Medically Relevant Fungi with the Host,” J. Fungi 7(10):875 (2021); Pantophlet et al., “Bacterially Derived Synthetic Mimetics of Mammalian Oligomannose Prime Antibody Responses that Neutralize HIV Infectivity,” Nat Commun 8:1601 (2017), each of which is hereby incorporated by reference in its entirety).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Specific aspects of the invention are identified below.

1. An oligosaccharide comprising three or more saccharide moieties, wherein two saccharide moieties at a non-reducing terminal end of the oligosaccharide are coupled together with a thio-ether bond, and one of the saccharide moieties at a reducing end of the oligosaccharide is coupled to a reactive moiety.

2. The oligosaccharide according to claim 1, wherein the reactive moiety is selected from the group consisting of an azido group, an alkynyl group, an alkenyl group, a thiol group, an amino group, or a carboxylic acid group.

3. The oligosaccharide according to claim 1 further comprising a linker between the one saccharide moiety and the reactive moiety.

4. The oligosaccharide according to claim 3, wherein the linker is —C₃-C₈ cycloalkyl-
—(CH₂)ₙ—, —S—(CH₂)ₙ—, —O—(CH₂)ₙ—, —(CH₂)ₘ—S—(CH₂)ₙ—, —(CH₂)ₘ—O—(CH₂)ₙ—, —O- aryl, or —S-aryl, wherein n and m are independently an integer from 1 to 20, preferably 2 to 10 or 2 to 4.

5. The oligosaccharide according to one of claims 1 to 4, wherein the two or more saccharide moieties comprise from two to twenty saccharide moieties.

6. The oligosaccharide according to claim 5, wherein four to twelve saccharide moieties are present.

7. The oligosaccharide according to claim 6, wherein the oligosaccharide is a branched oligosaccharide and the thio-ether bond is present between two saccharide moieties at a non reducing terminal end of each branch in the oligosaccharide.

8. The oligosaccharide according to claim 1, wherein the saccharide moieties are selected from the group consisting of glucose, galactose, mannose, arabinose, fucose, rhamnose, sialic acid, N-acetyl-glucosamine, and combinations thereof.

9. The oligosaccharide according to claim 1, wherein the oligosaccharide is

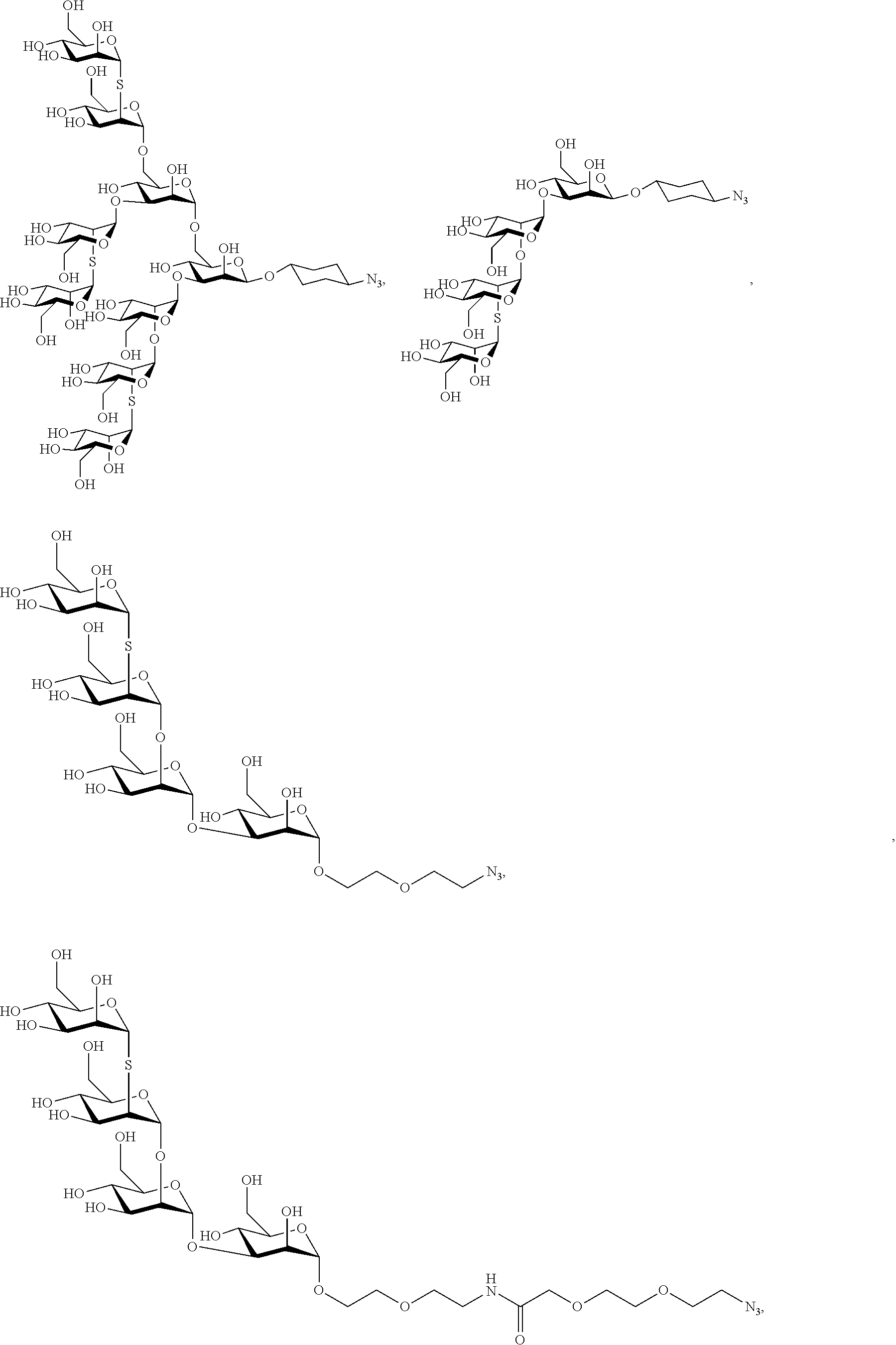

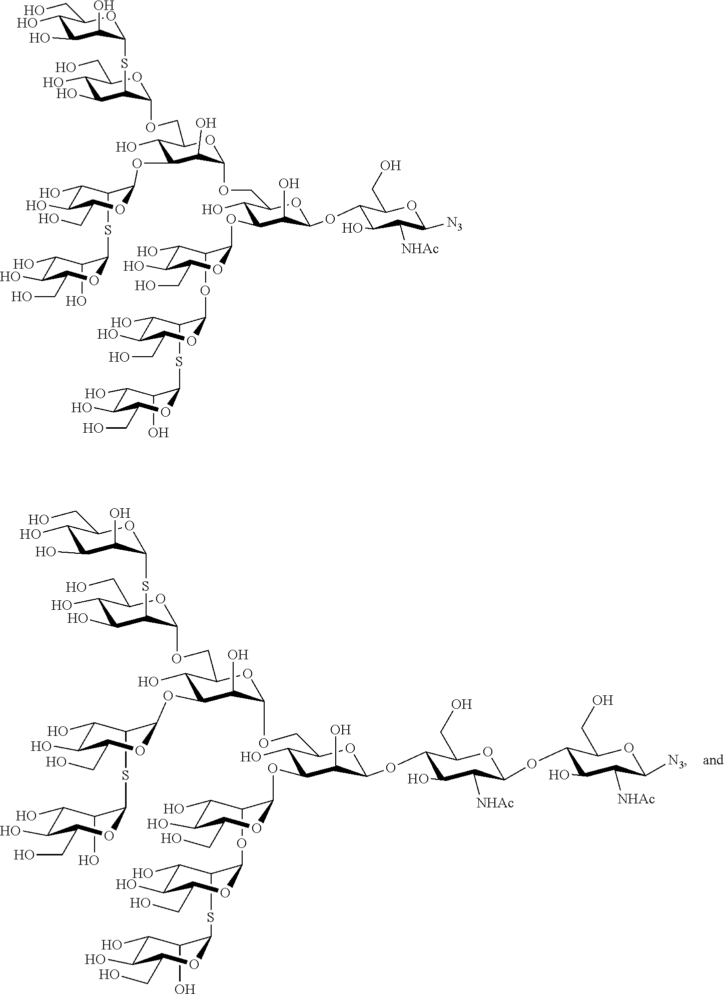

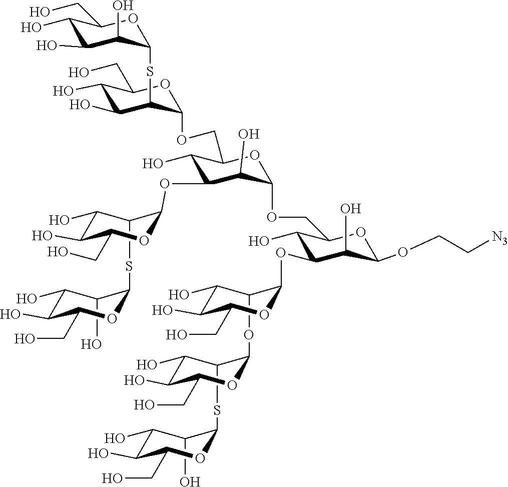

10. A glycopeptide comprising the oligosaccharide of claim 1 covalently linked to a sidechain of at least one amino acid residue in the peptide.

11. (canceled)

12. (canceled)

13. The glycopeptide according to claim 10, wherein the amino acid residue is a homopropargylglycine residue, which is reactive with an azido moiety of the oligosaccharide.

14. (canceled)

15. (canceled)

16. (canceled)

17. (canceled)

18. (canceled)

19. (canceled)

20. A glycosylated oligonucleotide molecule comprising at least one non-natural deoxynucleoside to which is covalently linked to the oligosaccharide of claim 1.

21. (canceled)

22. The oligonucleotide according to claim 20, wherein the non-natural deoxynucleoside is a substituted deoxyuridine.

23. (canceled)

24. (canceled)

25. (canceled)

26. (canceled)

27. (canceled)

28. (canceled)

29. (canceled)

30. (canceled)

31. (canceled)

32. An immunogenic conjugate comprising a glycopeptide according to claim 10 bound to an immunogenic carrier molecule.

33. (canceled)

34. (canceled)

35. (canceled)

36. (canceled)

37. (canceled)

38. A method of detecting a carbohydrate-binding antibody comprising: contacting a sample with the glycopeptide according to claim 10; and detecting whether a carbohydrate-binding antibody in the sample binds to the glycopeptide or the oligonucleotide.

39. (canceled)

40. (canceled)

41. (canceled)

42. (canceled)

43. (canceled)

44. (canceled)

45. (canceled)

46. (canceled)

47. (canceled)

48. A method of preparing a reactive oligosaccharide comprising: reacting an intermediate disaccharide having the structure 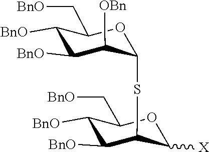
where X is a
leaving group, and is either in an alpha or beta orientation, with an acceptor monosaccharide or oligosaccharide that is partially protected and comprises at least one unprotected hydroxyl group, said reacting being carried out under conditions suitable to cause the disaccharide to covalently bond to the at least one unprotected hydroxyl group to form an oligosaccharide comprising three or more saccharide moieties, wherein two saccharide moieties at a non-reducing terminal end of the oligosaccharide are coupled together with a thio-ether bond, and one of the saccharide moieties at a reducing end of the oligosaccharide is coupled to a reactive moiety.

49. The method according to claim 48 further comprising deprotecting the oligosaccharide to remove protecting groups and expose hydroxyl groups in the oligosaccharide.

50. The method according to claim 48, wherein the leaving group is a halo group or a sulfonate group.

51. The method according to claim 48, wherein the leaving group has a beta orientation.

52. The oligosaccharide according to claim 1, comprising an alpha S-2-linked dimannose moiety
